(19) United States
(12) Patent Application Publication
(10) Pub. No.: US 2021/0236837 A1
(43) Pub. Date: Aug. 5, 2021

(54) TRANSCUTANEOUS ELECTRICAL AND/OR MAGNETIC SPINAL STIMULATION FOR BLADDER OR BOWEL CONTROL IN SUBJECTS WITHOUT CNS INJURY

(71) Applicant: The Regents of the University of California, Oakland, CA (US)

(72) Inventor: Daniel C. Lu, Los Angeles, CA (US)

(73) Assignee: The Regents of the University of California, Oakland, CA (US)

(21) Appl. No.: 17/269,970

(22) PCT Filed: Aug. 21, 2019
(86) PCT No.: PCT/US2019/047551

§ 371 Date: Feb. 19, 2021

Related U.S. Application Data
(60) Provisional application No. 62/827,782, filed on Apr. 1, 2019.
(60) Provisional application No. 62/720,835, filed on Aug. 21, 2018.

(51) Int. Cl. A61N 2/00; A61N 2/02

(52) U.S. Cl. CPC A61N 2/006; A61N 2/02

(57) ABSTRACT

In various embodiments methods and devices are provided for facilitating locomotor function and/or voiding of bladder and/or bowel in a subject with a neuromotor disorder. In certain embodiments the methods involve providing magnetic stimulation of the spinal cord at a location, frequency and intensity sufficient to facilitate locomotor function and/or voiding of bladder and/or bowel.

### CROSS-REFERENCE TO RELATED APPLICATIONS

This application claims priority to and benefit of U.S. Ser. No. 62/827,782, filed on Apr. 1, 2019, and to U.S. Ser. No. 62/720,835, filed on Aug. 21, 2018, both of which are incorporated herein by reference in their entirety.

### STATEMENT OF GOVERNMENTAL SUPPORT

This invention was made with government support under Grant Number W81XWH-14-2-0129, awarded by the U.S. Army, Medical Research and Materiel Command. The government has certain rights in the invention.

### BACKGROUND

There are numerous instances where subjects have impaired bladder and/or bowel function where the subject does not have a spinal cord or brain injury. For example, constipation is very common during pregnancy and occurs in about 50% of all pregnant women. Typically, for a pregnant woman, constipation is related to an increase in the hormone progesterone which slows the digestive process resulting in constipation, gas and heartburn. In addition the colon absorbs more water which makes stools harder. Worry, anxiety, minimal physical exercise, and a low-fiber diet may also cause constipation. Sometimes iron tablets may contribute to constipation.

Constipation is also common after surgery. Numerous factors may contribute to constipation after surgery and such factors may include, but are not limited to the use of narcotic pain relievers, such as opioids, general anesthesia, an inflammatory stimulus, such as trauma or infection, an electrolyte, fluid, or glucose imbalance, prolonged inactivity, and changes to diet, especially insufficient fiber.

Incontinence is also common. Seven major types of incontinence are: 1) Stress Incontinence; 2) Overflow incontinence; 3) Urge Incontinence or overactive bladder; 4) Functional incontinence; 5) Mixed incontinence; 6) Total Incontinence; and 7) Bedwetting.

Stress incontinence is related to pressure to urinary bladder such as overweight, pregnancy, sneezing, lifting heavy objects, exercise and some medical conditions. Stress incontinence is associated with increases in intrabdominal pressure (e.g., during a cough) that causes the involuntary release of urine through the urethra. Most cases are due to pelvic relaxation or insufficient support from the pelvic fascia and muscles with a hypermobile bladder neck causing unequal pressures between the bladder and the urethra. Risk factors include vaginal births, age, genetic predisposition, conditions causing chronic increased abdominal pressure, and conditions causing urethral weakening.

People with overflow incontinence usually have difficulties emptying their urinary bladder. Overflow incontinence most often affects men. Overflow incontinence can be due to decreased or no tone in the detrusor bladder muscle and may result in weak contractions and cause urinary retention. This, in turn, will cause the bladder to become overdistended and, once full, incontinence may occur. Obstruction may also cause similar symptoms.

Urge incontinence (e.g., detrusor instability) is characterized by an urge to urinate that is so strong that the patient has problems reaching to the toilet in time. Urge incontinence occurs in about 10-15% of the population and is due to involuntary contractions of the muscle within the bladder wall. The cause is often unknown but may be caused by any stimulus to receptors in the bladder wall (Infections, Stones, Foreign bodies, Bladder cancer, Suburethral diverticula) or neurologic disease (stroke, Alzheimer's, Parkinson's, Multiple Sclerosis, Diabetes).

Urine leaking associated with functional incontinence most often affects the elderly suffering from physical or/and mental diseases such as Alzheimer's disease and arthritis preventing them from reaching the toilet in time.

Mixed incontinence refers to urine leakage due to two or more types of incontinence simultaneously, most often due to overactive bladder and stress incontinence. Mixed incontinence typically affects women.

Total incontinence is the severest type of incontinence and is marked by complete loss of control over urinary bladder resulting in a constant urine leakage. Total incontinence can be caused when a urinary fistula forms between the bladder and the vagina, permitting urine to leak out continuously at all times. This is often due to previous radiation or surgery, but can be due to childbirth complications.

Bedwetting is a type of incontinence typically seen in children and is most often a result of the immaturity of the urinary bladder. Bedwetting in young children (by about the age of 5 years) is normal, while occasional “night accidents” in older children usually are not a cause of concern either. But if bedwetting persists, it is necessary to seek medical attention because in rare cases, it can be a sign of an underlying medical condition.

Without being bound to a particular theory, it is believed the methods and devices can be used to treat any of these forms of incontinence and/or constipation.

### SUMMARY

Recently, epidural spinal cord stimulation (SCS) was used to enhance motor function in individuals with chronic SCI (see, e.g., Harkema et al. (2011) Lancet, 377: 1938-1947; Angeli et al. (2014) Brain: J. Neurol. 137: 1394-1409; Lu et al. (2016) Neurorehabil. Neural Repair, 30: 951-962. We believe that spinal networks have the capacity to execute a range of complicated movements requiring detailed coordination among motor pools within the spine with minimal or even no input from the brain Lu et al. (2016) Neurorehabil. Neural Repair, 30: 951-962), and electrical or magnetic stimulation of the spine restores or permits coordinated activation of these spinal circuits. We hypothesized that a similar mechanism of SCS to the restoration of reaching and grasping function may be at play with respect to bladder function whereby co-contraction of agonist-antagonist muscles is abolished and voluntary motor control of micturition may be restored (Alam et al. (2017) Exp. Neurol., 291: 141-150). Thus, SCS can be used to address detrusor-sphincter dyssnergia (DSD), where there is agonist/antagonist muscle co-contraction, and disinhibit or enable volitional control of the spinal micturition circuit that coordinates detrusor constriction with sphincter relaxation.

Magnetic stimulation can be used to modulate neural circuits, and with figure-eight coils, the energy can be targeted to some extent. Moreover, transcutaneous magnetic stimulation is non-invasive and painless. Transcranial magnetic stimulation (TMS) has been used to modulate neuronal function in a variety of settings from migraine treatment (Zhu & Marmura (2016) Curr. Neurol. Neurosci. Rep. 16: 11) to depression (Perera et al. (2016) Brain. 9: 336-346) to restoration of motor function after ischemic stroke (Kim et al. (2016) J. Stroke, 18: 220-226). We used transcutaneous magnetic spinal cord stimulation (TMSCS) to stimulate the lumbar spine to try to improve bladder function in five patients with SCI who were unable to urinate voluntarily. We hypothesized that neuromodulation of the spine using TMSCS would allow these patients to achieve voluntary micturition and reduce or eliminate the need for bladder self-catheterization.

In view of the success with restoration of bladder and/or bowel function in subjects with a spinal cord injury, it is believe the same approach can be taken in subject that do not have a spinal cord or brain injury.

Accordingly, in various embodiments methods and devices are provided to restore the function of bladder or bowel in functions where voluntary control over bladder and/or bowel is impaired.

Various embodiments contemplated herein may include, but need not be limited to, one or more of the following:

Embodiment 1: A method of facilitating voiding or control of bladder and/or bowel in a subject with dysfunctional bladder and/or bowel function where said subject does not have a spinal cord or brain injury, said method comprising:
providing magnetic stimulation of the spinal cord at a location, frequency and intensity sufficient to facilitate voiding or control of bladder and/or bowel.

Embodiment 2: The method of embodiment 1, wherein said dysfunctional bladder and/or bowel comprises neurogenic bladder dysfunction.

Embodiment 3: The method of embodiment 1, wherein said dysfunctional bladder and/or bowel comprises post-surgical constipation.

Embodiment 4: The method of embodiment 1, wherein said dysfunctional bladder and/or bowel comprises narcotic-induced constipation.

Embodiment 5: The method of embodiment 4, wherein said dysfunctional bladder and/or bowel comprises opioid constipation.

Embodiment 6: The method of embodiment 1, wherein said dysfunctional bladder and/or bowel comprises dysfunction induced by an inflammatory stimulus, such as trauma or infection.

Embodiment 7: The method of embodiment 1, wherein said dysfunctional bladder and/or bowel comprises pregnancy associated bladder and/or bowel dysfunction.

Embodiment 8: The method of embodiment 1, wherein said dysfunctional bladder and/or bowel is associated with a condition selected from the group consisting of Meningomyelocele, Diabetes, AIDS, Alcohol abuse, Vitamin B12 deficiency neuropathies, Herniated disc, damage due to pelvic surgery, Syphilis, and a tumor.

Embodiment 9: The method according to any one of embodiments 1-8, wherein said method comprises facilitating voiding or control of bladder and/or bowel by providing magnetic stimulation of the spinal cord at a location, frequency and intensity sufficient to facilitate voiding or control of the bladder and/or bowel.

Embodiment 10: The method according to any one of embodiments 1-9, wherein said magnetic stimulation comprises stimulation at a frequency ranging from about 0.5 Hz up to about 15 Hz to induce micturition.

Embodiment 11: The method of embodiment 10, wherein said magnetic stimulation is at a frequency of about 1 Hz.

Embodiment 12: The method according to any one of embodiments 1-9, wherein said magnetic stimulation comprises stimulation at a frequency from about 20 Hz up to about 100 Hz to stop or prevent micturition.

Embodiment 13: The method of embodiment 12, wherein said magnetic stimulation is at a frequency of about 30 Hz.

Embodiment 14: The method according to any one of embodiments 1-13, wherein said magnetic stimulation comprises magnetic pulses ranging in duration from about 5 μs, or from about 10 μs, or from about 15 μs, or from about 20 μs up to about 500 μs, or up to about 400 μs, or up to about 300 μs, or up to about 200 μs, or up to about 100 μs. or up to about 50 μs.

Embodiment 15: The method of embodiment 14, wherein said magnetic pulses are about 25 μs in duration.

Embodiment 16: The method according to any one of embodiments 1-15, wherein said magnetic stimulation is monophasic.

Embodiment 17: The method according to any one of embodiments 1-16, wherein a single treatment of said magnetic stimulation comprises 1, or 2, or 3, or 4, or 5, or 6, or 7, or 8, or 9, or 10 or more continuous stimulation periods.

Embodiment 18: The method of embodiment 17, wherein a single treatment of said magnetic stimulation comprises about 3 continuous stimulation periods.

Embodiment 19: The method according to any one of embodiments 17-18, wherein said continuous stimulation periods range in duration from about 10 sec, or from about 20 sec, or from about 3 sec or from about 40 sec, or from about 50 sec, or from about 1 min, or from about 2 minutes up to about 10 minutes, or up to about 8 minutes, or up to about 6 minutes.

Embodiment 20: The method of embodiment 19, wherein said continues stimulation periods are about 4 minutes in duration.

Embodiment 21: The method according to any one of embodiments 17-20, wherein a delay between continuous stimulation periods ranges from about 5 sec, or from about 10 sec, or from about 15 sec, or from about 20 sec up to about 5 minutes, or up to about 4 minutes, or up to about 3 minutes, or up to about 2 minutes, or up to about 1 min, or up to about 45 sec, or up to about 30 sec.

Embodiment 22: The method of embodiment 21, wherein a delay between continuous stimulation periods is about 30 sec.

Embodiment 23: The method according to any one of embodiments 17-22, wherein said treatment is repeated.

Embodiment 24: The method of embodiment 23, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days.

Embodiment 25: The method according to any one of embodiments 23-24, wherein the treatment is repeated over a period of at least 1 week, or at least two weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 4 months, or at least 5 months, or at least 6 months, or at least 7 months, or at least 8 months, or at least 9 months, or at least 10 months, or at least 11 months, or at least 12 months.

Embodiment 26: The method according to any one of embodiments 1-25, wherein treatment of said subject with said magnetic stimulation facilitates volitional voiding at a later time without magnetic stimulation.

Embodiment 27: The method according to any one of embodiments 23-26, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains volitional control of micturation.

Embodiment 28: The method of embodiment 27, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains their maximal volitional control of micturation.

Embodiment 29: The method of embodiment 27, wherein the frequency of treatment is reduced after the subject obtains volitional control of micturition.

Embodiment 30: The method of embodiment 28, wherein the frequency of treatment is reduced after the subject obtains maximal volitional control of micturition.

Embodiment 31: The method according to any one of embodiments 29-30, wherein the frequency of treatment is reduced to a level sufficient to maintain volitional control of micturition.

Embodiment 32: The method of embodiment 31, wherein the frequency of treatment is reduced to every three days, or to a weekly treatment, or to about every 10 days, or to about every 2 weeks.

Embodiment 33: The method according to any one of embodiments 1-32, wherein said magnetic stimulation is applied over the thoracic and/or lumbosacral spinal cord.

Embodiment 34: The method of embodiment 33, wherein said magnetic stimulation is applied over one or more regions selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, T12-T12, L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6.

Embodiment 35: The method of embodiment 33, wherein said magnetic stimulation is applied over a region between T11 and L4.

Embodiment 36: The method of embodiment 35, wherein said magnetic stimulation is applied over one or more regions selected from the group consisting of T11-T12, L1-L2, and L2-L3.

Embodiment 37: The method of embodiment 35, wherein said magnetic stimulation is applied over L1-L2 and/or over T11-T12.

Embodiment 38: The method of embodiment 35, wherein said magnetic stimulation is applied over L1.

Embodiment 39: The method according to any one of embodiments 1-38, wherein said magnetic stimulation is applied at the midline of spinal cord.

Embodiment 40: The method according to any one of embodiments 1-39, wherein said magnetic stimulation produces a magnetic field of at least about 1 tesla, or at least about 2 tesla, or at least about 3 tesla, or at least about 4 tesla, or at least about 5 tesla.

Embodiment 41: The method according to any one of embodiments 1-9, or 17-40, wherein said magnetic stimulation is at a frequency of at least about 0.5 Hz, 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

Embodiment 42: A method of facilitating voiding or control of bladder and/or bowel in a subject with a dysfunctional bladder and/or bowel function where said subject does not have a spinal cord or brain injury, said method comprising:
providing transcutaneous electrical stimulation of the spinal cord at a location, frequency and intensity sufficient to facilitate voiding or control of bladder and/or bowel.

Embodiment 43: The method of embodiment 42, wherein said dysfunctional bladder and/or bowel comprises neurogenic bladder dysfunction.

Embodiment 44: The method of embodiment 42, wherein said dysfunctional bladder and/or bowel comprises post-surgical constipation.

Embodiment 45: The method of embodiment 42, wherein said dysfunctional bladder and/or bowel comprises narcotic-induced constipation.

Embodiment 46: The method of embodiment 45, wherein said dysfunctional bladder and/or bowel comprises opioid constipation.

Embodiment 47: The method of embodiment 42, wherein said dysfunctional bladder and/or bowel comprises dysfunction induced by an inflammatory stimulus, such as trauma or infection.

Embodiment 48: The method of embodiment 42, wherein said dysfunctional bladder and/or bowel comprises pregnancy associated bladder and/or bowel dysfunction.

Embodiment 49: The method of embodiment 42, wherein said dysfunctional bladder and/or bowel is associated with a condition selected from the group consisting of Meningomyelocele, Diabetes, AIDS, Alcohol abuse, Vitamin B12 deficiency neuropathies, Herniated disc, damage due to pelvic surgery, Syphilis, and a tumor.

Embodiment 50: The method according to any one of embodiments 42-49, wherein said method comprises facilitating voiding or control of bladder and/or bowel by providing transcutaneous electrical stimulation of the spinal cord at a location, frequency and intensity sufficient to facilitate voiding or control of the bladder and/or bowel.

Embodiment 51: The method according to any one of embodiments 42-50, wherein said transcutaneous electrical stimulation comprises stimulation at a frequency of at least about 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz, and/or at a frequency ranging from about 1 Hz, or from about 2 Hz, or from about 3 Hz, or from about 4 Hz, or from about 5 Hz, or from about 10 Hz, or from about 10 Hz, or from about 10 Hz, up to about 500 Hz, or up to about 400 Hz, or up to about 300 Hz, or up to about 200 Hz up to about 100 Hz, or up to about 90 Hz, or up to about 80 Hz, or up to about 60 Hz, or up to about 40 Hz, or from about 3 Hz or from about 5 Hz up to about 80 Hz, or from about 5 Hz to about 60 Hz, or up to about 30 Hz. In certain embodiments the transcutaneous stimulation is at a frequency ranging from about 20 Hz or about 30 Hz to about 90 Hz or to about 100 Hz.

Embodiment 52: The method according to any one of embodiments 42-51, wherein the transcutaneous electrical stimulation is provided on a high frequency carrier signal.

Embodiment 53: The method of embodiment 52, wherein the high frequency carrier signal ranges from about 3 kHz, or about 5 kHz, or about 8 kHz up to about 30 kHz, or up to about 20 kHz, or up to about 15 kHz.

Embodiment 54: The method according to any one of embodiments 52-53, wherein the carrier frequency amplitude ranges from about 30 mA, or about 40 mA, or about 50 mA, or about 60 mA, or about 70 mA, or about 80 mA up to about 300 mA, or up to about 200 mA, or up to about 150 mA.

Embodiment 55: The method according to any one of embodiments 52- 54, wherein said transcutaneous electrical stimulus is a high frequency stimulus at a duration ranging from about 0.1 up to about 2 ms, or from about 0.1 up to about 1 ms, or from about 0.5 ms up to about 1 ms, or for about 0.5 ms.

Embodiment 56: The method according to any one of embodiments 52-55, wherein the transcutaneous electrical stimulation comprises a 10 kHz stimulus repeated at 1-40 times per second.

Embodiment 57: The method according to any one of embodiments 42-56, wherein said transcutaneous electrical stimulus is applied for 1 to 30 s, or for about 5 to 30 s, or for about 10 to about 30 s.

Embodiment 58: The method according to any one of embodiments 42-57, wherein said transcutaneous electrical stimulus is about 30 to about 100 mA.

Embodiment 59: The method according to any one of embodiments 52-58, wherein said transcutaneous electrical stimulus comprises a 10 kHz signal applied at 1 Hz.

Embodiment 60: The method according to any one of embodiments 42-59, wherein said transcutaneous electrical stimulus comprises a constant-current bipolar rectangular stimulus.

Embodiment 61: The method according to any one of embodiments 42-60, wherein said transcutaneous electrical stimulation comprises pulses ranging in duration from about 5 μs, or from about 10 μs, or from about 15 μs, or from about 20 μs up to about 2 ms, or up to about 1 ms, or up to about 2 ms, or up to about 500 μs, or up to about 400 μs, or up to about 300 μs, or up to about 200 μs, or up to about 100 μs. or up to about 50 μs.

Embodiment 62: The method of embodiment 61, wherein said pulses are about 1 ms in duration.

Embodiment 63: The method according to any one of embodiments 42-62, wherein a single treatment of said transcutaneous electrical stimulation comprises 1, or 2, or 3, or 4, or 5, or 6, or 7, or 8, or 9, or 10 or more continuous stimulation periods.

Embodiment 64: The method of embodiment 63, wherein said treatment is repeated.

Embodiment 65: The method of embodiment 64, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days.

Embodiment 66: The method according to any one of embodiments 64-65, wherein the treatment is repeated over a period of at least 1 week, or at least two weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 4 months, or at least 5 months, or at least 6 months, or at least 7 months, or at least 8 months, or at least 9 months, or at least 10 months, or at least 11 months, or at least 12 months.

Embodiment 67: The method according to any one of embodiments 42-66, wherein treatment of said subject with said transcutaneous electrical stimulation facilitates volitional voiding at a later time without transcutaneous electrical stimulation.

Embodiment 68: The method according to any one of embodiments 64-67, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains volitional control of micturation.

Embodiment 69: The method according to any one of embodiments 64-67, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains their maximal volitional control of micturation.

Embodiment 70: The method according to any one of embodiments 64-67, wherein the frequency of treatment is reduced after the subject obtains volitional control of micturition.

Embodiment 71: The method according to any one of embodiments 64-67, wherein the frequency of treatment is reduced after the subject obtains maximal volitional control of micturition.

Embodiment 72: The method according to any one of embodiments 70-71, wherein the frequency of treatment is reduced to a level sufficient to maintain volitional control of micturition.

Embodiment 73: The method according to any one of embodiments 42-72, wherein said transcutaneous electrical stimulation is applied over one or more regions selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, T12-T12, L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6.

Embodiment 74: The method of embodiment 73, wherein said transcutaneous electrical stimulation is applied over a region between T11 and L4.

Embodiment 75: The method of embodiment 74, wherein said transcutaneous electrical stimulation is applied over one or more regions selected from the group consisting of T11-T12, L1-L2, and L2-L3.

Embodiment 76: The method of embodiment 74, wherein said transcutaneous electrical stimulation is applied over L1-L2 and/or over T11-T12.

Embodiment 77: The method of embodiment 74, wherein said transcutaneous electrical stimulation is applied over L1.

Embodiment 78: The method according to any one of embodiments 42-77, wherein said transcutaneous electrical stimulation is applied at the midline of spinal cord.

Embodiment 79: The method according to any one of embodiments 1-78, wherein said subject is a subject without a neurodegenerative pathology.

Embodiment 80: The method of embodiment 79, wherein said subject does not have Parkinson's disease, Huntington's disease, Alzheimer's disease, amyotrophic lateral sclerosis (ALS), primary lateral sclerosis (PLS), and/or cerebral palsy.

Embodiment 81: A method of facilitating voiding or control of bladder and/or bowel in a subject with dysfunctional bladder and/or bowel function where said subject does not have a spinal cord or brain injury, said method comprising: providing magnetic stimulation in combination with transcutaneous electrical stimulation at one or more locations, frequencies, and intensities sufficient to facilitate voiding or control of bladder and/or bowel.

Embodiment 82: The method of embodiment 81, wherein said method comprises providing magnetic stimulation to said subject using a method according to any one of embodiments 1-41 in combination with electrical stimulation using a method according to any one of embodiments 42-79.

### DEFINITIONS

As used herein “electrical stimulation” or “stimulation” means application of an electrical signal that may be either excitatory or inhibitory to a muscle or neuron and/or to groups of neurons and/or interneurons. It will be understood that an electrical signal may be applied to one or more electrodes with one or more return electrodes.

As used herein “magnetic stimulation” or means use of a varying magnetic field to induce an electrical signal, e.g., in a neuron, that may be either excitatory or inhibitory to a muscle or neuron and/or to groups of neurons and/or interneurons.

As used herein “epidural” means situated upon the dura or in very close proximity to the dura. The term “epidural stimulation” refers to electrical epidural stimulation. In certain embodiments epidural stimulation is referred to as “electrical enabling motor control” (eEmc).

The term “transcutaneous stimulation” or “transcutaneous electrical stimulation” or “cutaneous electrical stimulation” refers to electrical stimulation applied to the skin, and, as typically used herein refers to electrical stimulation applied to the skin in order to effect stimulation of the spinal cord or a region thereof. The term “transcutaneous electrical spinal cord stimulation” may also be referred to as “tSCS”. The term “pcEmc” refers to painless cutaneous electrical stimulation.

The term “motor complete” when used with respect to a spinal cord injury indicates that there is no motor function below the lesion, (e.g., no movement can be voluntarily induced in muscles innervated by spinal segments below the spinal lesion.

The term “monopolar stimulation” refers to stimulation between a local electrode and a common distant return electrode.

The term “co-administering”, “concurrent administration”, “administering in conjunction with” or “administering in combination” when used, for example with respect to transcutaneous electrical stimulation, epidural electrical stimulation, and pharmaceutical administration, refers to administration of the transcutaneous electrical stimulation and/or epidural electrical stimulation and/or pharmaceutical such that various modalities can simultaneously achieve a physiological effect on the subject. The administered modalities need not be administered together, either temporally or at the same site. In some embodiments, the various “treatment” modalities are administered at different times. In some embodiments, administration of one can precede administration of the other (e.g., drug before electrical and/or magnetic stimulation or vice versa). Simultaneous physiological effect need not necessarily require presence of drug and the electrical and/or magnetic stimulation at the same time or the presence of both stimulation modalities at the same time. In some embodiments, all the modalities are administered essentially simultaneously.

The phrase “spinal cord stimulation” as used herein includes stimulation of any spinal nervous tissue, including spinal neurons, accessory neuronal cells, nerves, nerve roots, nerve fibers, or tissues, that are associated with the spinal cord. It is contemplated that spinal cord stimulation may comprise stimulation of one or more areas associated with a cervical vertebral segment.

As used herein, “spinal nervous tissue” refers to nerves, neurons, neuroglial cells, glial cells, neuronal accessory cells, nerve roots, nerve fibers, nerve rootlets, parts of nerves, nerve bundles, mixed nerves, sensory fibers, motor fibers, dorsal root, ventral root, dorsal root ganglion, spinal ganglion, ventral motor root, general somatic afferent fibers, general visceral afferent fibers, general somatic efferent fibers, general visceral efferent fibers, grey matter, white matter, the dorsal column, the lateral column, and/or the ventral column associated with the spinal cord. Spinal nervous tissue includes “spinal nerve roots,” that comprise any one or more of the 31 pairs of nerves that emerge from the spinal cord. Spinal nerve roots may be cervical nerve roots, thoracic nerve roots, and lumbar nerve roots.

### BRIEF DESCRIPTION OF THE DRAWINGS

FIG. 1 shows a schematic illustration of one illustrative embodiment of a magnetic nerve stimulator.

FIG. 2. Overview of the study. There were three phases of the study: assessment, treatment and follow-up. The time frame for each is shown in the flow chart. During the assessment phase, each subject received stimulation with both 1 Hz and 30 Hz, each stimulation frequency delivered for one week, and underwent urodynamic testing (UDS) with video recording at the end of the assessment phase to determine the optimal frequency based on the changes in urethral and detrusor pressures during micturition attempts with either stimulating frequency. The 1 Hz stimulation frequency reduced urethral pressure and increased detrusor pressure in all subjects more effectively than 30 Hz stimulation. Therefore, each subject received 1 Hz stimulation during the treatment phase and received weekly stimulation treatment for 16 weeks. During the follow-up phase, the subject received “sham” stimulation at <5% intensity in order to blind each subject to the change in stimulation treatment. The follow-up phase lasted 6 weeks or until each subject's urological improvements completely dissipated.

FIG. 3, panels A-E, shows T2-weighted MRI imaging showing the degree of SCI in all five subjects enrolled in the study. The MRIs were obtained to ensure there was no spinal cord transection and to assess the anatomical level of injury (cervical/thoracic/lumbar). Authors reviewed all the MRIs prior to enrolling each the subject in the study. A synopsis of the formal neuroradiology report was reviewed and included here for reference. (A) Prominent metallic artifact from fusion hardware in the superior to midthoracic spine significantly obscures evaluation at these levels. The small segment in which the cord can be visualized at the T4-T5 demonstrates prominent cord myelomalacia. Stable compression deformity of T5 without retropulsion. Scattered discogenic changes are seen in the thoracic spine from T8 through T12 without significant foraminal or canal stenosis. The cord is unremarkable at these levels. (B) Metallic artifact from instrumentation hardware in upper thoracic spine makes the evaluation of the spinal cord difficult at high thoracic spine levels. On axial images, significant myelomalacia is noted at T3-4 level. Below T5, the spinal cord appears to have normal caliber. No significant canal or foraminal stenosis. (C) Severe spinal cord myelomalacia at C5-C6. No evidence of spinal cord edema. Grossly stable anterior and posterior fusion from C4 to C6. Left vertebral artery occlusion, possibly related to chronic traumatic dissection. (D) Status post anterior fusion from C5 to C7 and posterior fusion. Metallic distortion artefact is noted through the fused C5 to C7 levels and significant myelomalacia or cord edema is noted at these levels. Visualized upper thoracic spinal cord appears to be in normal caliber with no compression. (E) Status post ACDF from C6 to T1 for repair of C7 burst fracture. Spinal cord edema and swelling spans from C4-T1.

FIG. 4, panels A-C, shows an example of the BCR amplitude (A), which is measured from the perineal muscle EMG activity, obtained from subject C at baseline and during low frequency (1 Hz) and high frequency (30 Hz) TCSMS of the lumbar spine at the end of the assessment phase of the study. The BCR was elicited serially >100 times, and the mean (solid black line) ±2 times the SD (cyan shading) are shown for each stimulation condition. The average and standard deviation of BCR responses to 1 and 30 Hz TCSMS (B), expressed as a percent of the baseline value in each subject, are shown to illustrate that the BCR amplitude was significantly reduced during 1 Hz TCSMS compared to 30 Hz TCSMS. Student's t-test: *p<0.0001, n.s.=non-significant, N=100 BCR cycles. BCR=bulbocavernosus reflex. Examples of evoked EMG activity from a single subject in selected muscles are shown in C. Lumbar TCSMS at 1 Hz elicited significant EMG activity, but 30 Hz TCSMS did not alter EMG activity. Ensemble averages of EMG activity (solid black line)±2 times the SD (cyan shading) were derived from greater than 100 cycles of stimulation. The stimulus artifacts are shown in the 30 Hz stimulation sequences since stimulation occurred multiple times within the recording window (large black spikes). The left (L) perineum, left vastus lateralis, right (R) vastus lateralis and left quadriceps femoris muscles were recorded. The arrows in panel A represent the peak and the nadir of the BCR.

FIG. 5, panels A-D. Examples of video urodynamics are shown from patient A (panel A—before the 16-week TMSCS treatment and panel D—after the 16 week TMSCS treatment). The first video images in each sequence show the pre-voiding bladder capacity, which increased after TMSCS. The second images show the initiation of volitional voiding and opening the bladder neck (white arrows), and the final images show the post-void residuals. In panel B, examples of urine flow (red line); urethral pressure (black line) and detrusor pressure (blue line) are shown before (upper graph) and after the 16-week TCSMS treatment (lower graph). Note that detrusor pressure remained below urethral pressure before TMSCS, and no urine flow was generated; whereas detrusor pressure exceeded urethral pressure and urine flow was generated after 16 weeks of TMSCS. The average urethral and detrusor pressures±SD obtained during efforts to void at the end of the assessment phase are shown in panel C during baseline and 1 and 30 Hz TMSCS. The detrusor pressure rose significantly and the urethral pressure fell significantly only during 1 Hz TMSCS compared to the non-stimulated condition and the 30 Hz condition (**p<0.0001), but the baseline, unstimulated state and the 30 Hz condition did not differ from each other based on an ANOVA and specific comparisons using Tukey's HSD.

FIG. 6 shows a summary of urological functions for all five subjects and average daily volitional micturition volume for all five subjects during follow-up phase; all changes were statistical significant when tested with paired t-tests (p<0.05; see Results for details). The top panel shows the timing of recovery and loss of voluntary control of micturition and the volume of urine produced each day as a function of time. All five subjects recovered the capacity to urinate voluntarily, and about 2-3 weeks after the termination of TMSCS, the capacity to urinate voluntarily declined rapidly back to the baseline (unable to void voluntarily). The remaining panels indicate the initial value of each variable before the start of TMSCS and after 16 weeks of TMSCS. The urine stream velocity and the bladder capacity (both measured during urodynamic studies) increased significantly (p<0.05) after 16 weeks of TMSCS. The residual volume and the number of self-catheterizations diminished significantly (p<0.05, for both variables), and the SHIM Score and the iQOL, both quality of life measures, increased significantly (p<0.05) after 16 weeks of TMSCS.

FIG. 7 illustrates bladder volume of post-operative opioid-induced urinary retention patient treated with non-invasive magnetic spinal cord stimulation.

FIG. 8 illustrates voiding efficiency in 4 patients with opioid-induced urinary retention treated with non-invasive magnetic spinal cord stimulation.

FIG. 9 illustrates the results of an assessment of incontinence in subjects treated with non-invasive magnetic spinal cord stimulation.

FIG. 10 illustrates time to bowel sounds and bowel movement in post-operative patients treated with magnetic stimulation at conus compared to sham treated patients.

FIG. 11 shows that magnetic stimulation decreases the length of post-operative hospitalization.

### DETAILED DESCRIPTION

In various embodiments methods and devices are provided to facilitate bladder and/or bowel control in subjects that have dysfunctional bladder or bowel control where there is no brain or spinal cord injury. In certain embodiments the dysfunctional bladder and/or bowel comprises neurogenic bladder dysfunction. In certain embodiments the dysfunctional bladder and/or bowel comprises dysfunction induced by an inflammatory stimulus, such as trauma or infection. In certain embodiments the dysfunctional bladder and/or bowel comprises pregnancy associated bladder and/or bowel dysfunction. In certain embodiments the dysfunctional bladder and/or bowel is associated with a condition selected from the group consisting of Meningomyelocele, Diabetes, AIDS, alcohol abuse, Vitamin B12 deficiency neuropathies, herniated disc, damage due to pelvic surgery, syphilis, a tumor, and the like. It will be recognized that these examples are illustrative and the methods and devices described herein can be used to facilitate bladder and/or bowel function associated with essentially any dysfunctional state.

In certain embodiments, the dysfunctional bowel comprises constipation induced by one or more medical procedures, one or more drugs, one or more disorders, etc. For example, in certain embodiments the dysfunctional bowel may comprise post-surgical constipation. As another example, in certain embodiments the dysfunctional bowel may be induced by one or more medications (e.g., opiates (e.g., morphine) or other narcotics, anticholinergic agents, tricyclic antidepressants (amitriptyline), antispasmodics (dicyclomine, mebeverine, peppermint oil), calcium channel blockers (verapamil, nifedipine), antiparkinsonian drugs, anticonvulsants (carbamazepine), sympathomimetics (ephedrine), antipsychotics (chloropromazine, clozapine, haloperidol, risperidone), diuretics (furosemide), antihypertensives (clonidine), antiarrhythmics (amiodarone), beta-adrenoceptor antagonists (atenolol), antihistamines, calcium or aluminum containing antacids, calcium supplements, iron supplements, antidiarrheal (loperamide), 5-HT3-receptor antagonists (ondansetron), bile acid sequestrants (cholestyramine), non-steroidal anti-inflammatory drugs (ibuprofen), etc.). As yet another example, in certain embodiments, the dysfunctional bowel may comprise a condition that is secondary to a primary disease or disorder such as organic stenosis (e.g., colorectal cancer or other intra- or extra-intestinal masses, inflammatory stenosis, ischemic stenosis, surgical stenosis, etc.), an endocrine or metabolic disorder (e.g., hypothyroidism, hypercalcemia, hyperparathyroidism, diabetes, porphyria, chronic renal insufficiency, panhypopituitarism, pregnancy, etc.), neurological disorders (e.g., Parkinson's disease, cerebrovasular disease, paraplegia, multiple sclerosis, autonomic neuropathy, spina bifida, etc.), an enteric neuropathy (e.g., Hirschsprung's disease, chronic intestinal pseudo-obstruction, etc.), a myogenic disorder (e.g., myotonic dystrophy, dermatomyositis, scleroderma, amyloidosis, chronic intestinal pseudo-obstruction, etc.), an anorectal disorder (e.g., anal fissures, anal strictures, etc.), and the like.

In certain embodiments, the dysfunctional bowel comprises one or more diarrheal conditions. For example, in certain embodiments, the dysfunctional bowel may comprise an acute diarrheal condition or a chronic diarrheal condition. In certain embodiments, diarrheal condition may be caused by a microbe (e.g., viral gastroenteritis, such as caused by rotavirus, norovirus, etc., or bacteria). In certain embodiments, the dysfunctional bowel may comprise fatty or malabsorptive diarrhea, which may, for example, be due to chronic pancreatitis or other chronic injury to the pancreas (e.g., alcohol damage, cystic fibrosis, hereditary pancreatitis, pancreatic cancer, other trauma to the pancreas, etc.) and/or small bowel disease (e.g., celiac disease, Crohn's disease, Whipple's disease, tropical sprue, eosinophilic gastroenteritis, etc.). In certain embodiments, the dysfunctional bowel may comprise a watery diarrheal condition, such that caused by carbohydrate malabsorption (e.g., intolerance to lactose, sorbitol, fructose, etc.). In certain embodiments, the dysfunctional bowel may comprise medication-induced diarrhea such as induced by antibiotics, NSAIDs, antacids, antihypertensives, antiarrhythmics, etc. In certain embodiments, the dysfunctional bowel may comprise diarrhea due to inflammatory bowel disease (IBD), ulcerative colitis, Crohn's disease ischemia of the gut, infections, a medical procedure (e.g., radiation therapy), colon cancer, polyps, irritable bowel syndrome (IBS), diabetes mellitus, other chronic medical conditions, diet, etc.

It was discovered that stimulation with devices that impart a magnetic field (e.g., at a frequency range from about 0.5 Hz up to about 100 Hz) can regulate bladder function. In particular, low frequency magnetic stimulation (e.g., 0.5 Hz up to about 20 Hz) can induce micturition, while hither frequency magnetic stimulation (e.g. 20 Hz or 30 Hz up to about 10 Hz or 100 Hz) can suppress micturition. More surprisingly it was discovered that repeated treatments with magnetic stimulation can over time increase volitional control of bladder function. Once volitional control of bladder function is realized, repeated periodic treatments (e.g., weekly, every 10 days, biweekly, etc.) can maintain this volitional bladder control.

It was also discovered that stimulation with devices that impart an electrical or magnetic field (e.g., at a frequency range from 5-100 Hz) of the cervical, and/or thoracic, and/or lumbar spinal cord, nerve roots, or combinations thereof can restore arm and leg movement (e.g., in subjects with a partial or full spinal cord injury). It was also discovered that, with training and repetition, the gains with stimulation can be hardwired and present even without stimulation. Additionally, it was discovered that serotonin agonists such as buspirone and the like can be used to further activate the spinal network to improve motor function.

Stimulation of the cervical, and/or thoracic, and/or lumbar spinal cord, nerve roots, or combinations thereof can be induced by epidural stimulation electrodes, non-invasive transcutaneous electrical stimulation, or magnetic stimulation.

Additionally, it was discovered that the stimulation methods described herein can be leveraged to regain motor function in subjects with injury to the central nervous system or degenerative neuromotor conditions, including, but not limited to stroke, TBI, MS, ALS, Parkinson's disease, Alzheimer's disease, and the like.

Without being bound to a particular theory, it is believed that enabling the spinal circuitry can produce a coordinated behavior that is more complete and physiologic than stimulation of individual nerve roots or the peripheral nerves. Moreover, the existing devices have the disadvantages of being invasive, producing a subset of the desired locomotor or micturition behavior, and do not result in enduring plastic changes to the circuitry that allow patients to become device independent.

By way of illustration, it is noted that Medtronic markets the INTERSTIM® device for sacral neuromodulation with overactive bladder or fecal incontinence. This device can be effective, but there is a fundamental difference in the mechanism of action compared to the methods described herein. Neuromodulation of the sacral nerve roots, as with the Medtronic InterStim, attempts to produce appropriate behavior by altering the activity of the sacral nerves.

In contrast, the methods described herein alter the activity of the spinal circuitry. It is believed that enabling the spinal circuitry produces a coordinated behavior that is more complete and physiologically normative than stimulation of the peripheral nerves. Moreover, the existing devices have the disadvantages of being invasive, producing a subset of the micturition behavior, and do not result in enduring plastic changes to the circuitry that allow patients to become device independent.

### Voiding of Bladder and/or Bowel

As explained above, the orchestrated neuromuscular control of urinary bladder function by the sensory, motor and autonomic nervous systems can be impaired by degenerative or traumatic changes, such as multiple sclerosis, spinal cord injury, stroke. It was discovered that stimulation of the spinal cord and, optionally, associated nerve roots can restore voluntary control of bladder and/or bowel function.

In particular, it was discovered that non-invasive (e.g., magnetic or transcutaneous electrical) stimulation of the cervical, thoracic, lumbar (vertebral body designation) spinal cord and associated nerve roots and combination thereof, results in micturition and/or restoration of bowel function. In particular it was observed that electrical stimulation with (10 kHz constant-current bipolar rectangular stimulus) from a range of 1 Hz to 100 Hz enabled micturition and restoration of bowel function. It was also observed that stimulation with a magnetic stimulator, generating a magnetic field, within a range of 1 Hz to 100 Hz enabled micturition and restoration of bowel function.

### Magnetic Stimulation to Restore Bladder/Bowel Function

More generally, it was discovered that that stimulation of the spinal cord with devices that impart a magnetic field (e.g., at a frequency range from about 0.5 Hz up to about 100 Hz) can regulate bladder function. In particular, low frequency magnetic stimulation (e.g., 0.5Hz up to about 15 Hz) can induce micturition, while higher frequency magnetic stimulation (e.g. 20 Hz or 30 Hz up to about 100 Hz) can suppress micturition. Thus, for example, it was observed that at a low frequency (e.g., 1 Hz) the detrusor pressure increased with minimal or small change in urethral pressure so micturition seemed to be enhanced (which can be used to treat underactive and neurogenic bladder). At high frequency (e.g., 30 Hz) urethreal pressure increased with no modification of detrusor pressure so urine can be retained (which can be used to treat overactive bladder or stress incontinence).

More surprisingly it was discovered that repeated treatments with magnetic stimulation can over time increase volitional control of bladder function. Once volitional control of bladder function is realized, repeated periodic treatments (e.g., weekly, every 10 days, biweekly, etc.) can maintain this volitional bladder control.

Accordingly, in various embodiments methods of facilitating voiding or control of bladder and/or bowel in a subject with a neuromotor disorder are provided where the methods involve providing magnetic stimulation of the spinal cord at a location, frequency and intensity sufficient to facilitate voiding or control of bladder and/or bowel. In certain embodiments the magnetic stimulation comprises stimulation at a frequency ranging from about 0.5 Hz up to about 15 Hz to induce micturition and in certain embodiments the magnetic stimulation is at a frequency of about 1 Hz. In certain embodiments the magnetic stimulation comprises stimulation at a frequency from about 20 Hz up to about 100 Hz to stop or prevent micturition and in certain embodiments, the magnetic stimulation is at a frequency of about 30 Hz.

In certain embodiments the magnetic stimulation comprises magnetic pulses ranging in duration from about 5 μs, or from about 10 μs, or from about 15 μs, or from about 20 μs up to about 1 ms, or up to about 750 μs, or up to about 500 μs, or up to about 400 μs, or up to about 300 μs, or up to about 200 μs, or up to about 100 μs. or up to about 50 μs. In certain embodiments the magnetic pulses are about 25 μs in duration.

In certain embodiments the magnetic stimulation is monophasic, while in other embodiments, the magnetic stimulation is biphasic.

In certain embodiments a single treatment of magnetic stimulation comprises 1, or 2, or 3, or 4, or 5, or 6, or 7, or 8, or 9, or 10 or more continuous stimulation periods. In various embodiments the continuous stimulation periods range in duration from about 10 sec, or from about 20 sec, or from about 3 sec or from about 40 sec, or from about 50 sec, or from about 1 min, or from about 2 minutes up to about 10 minutes, or up to about 8 minutes, or up to about 6 minutes. In certain embodiments the continuous stimulation periods are about 4 minutes in duration. In certain embodiments the delay between continuous stimulation periods ranges from about 2 sec, or from about 5 sec, or from about 10 sec, or from about 15 sec, or from about 20 sec up to about 5 minutes, or up to about 4 minutes, or up to about 3 minutes, or up to about 2 minutes, or up to about 1 min, or up to about 45 sec, or up to about 30 sec. In certain embodiments the delay between continuous stimulation periods is about 30 sec.

It was discovered that repeating the treatment can progressively increase subsequent volitional control of bladder function (e.g., permits volitional voiding at a later time without magnetic (or electrical) stimulation). Conversely removal of repetitive treatments can result in progressive loss of volitional control. Accordingly, in certain embodiments the treatment is repeated (e.g., repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days). In certain embodiments the treatment is repeated over a period of at least 1 week, or at least two weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 4 months, or at least 5 months, or at least 6 months, or at least 7 months, or at least 8 months, or at least 9 months, or at least 10 months, or at least 11 months, or at least 12 months. In certain embodiments the treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains volitional control of micturation. In certain embodiments the treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains their maximal volitional control of micturation.

In certain embodiments, once volitional control is achieved, the frequency of treatment can be reduced to a “maintenance” level. Typically, the frequency of treatment is is reduced to a level sufficient to maintain volitional control (e.g., a desired level of volitional control) of micturition. In certain embodiments the frequency of treatment is reduced to every three days, or to a weekly treatment, or to about every 10 days, or to about every 2 weeks.

In certain embodiments the magnetic stimulation is applied over the thoracic and/or lumbosacral spinal cord (e.g., over one or more regions selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, T12-T12, L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6). In certain embodiments the magnetic stimulation is applied over a region between T11 and L4. In certain embodiments the magnetic stimulation is applied over one or more regions selected from the group consisting of T11-T12, L1-L2, and L2-L3. In certain embodiments the magnetic stimulation is applied over L1-L2 and/or over T11-T12. In certain embodiments the magnetic stimulation is applied over L1. In certain embodiments the magnetic stimulation is applied at the midline of spinal cord. In various embodiments the magnetic stimulation produces a magnetic field of at least about 0.5 tesla, or at least about 0.6 tesla, or at least about 0.7 tesla, or at least about 0.8 tesla, or at least about 0.9 tesla, or at least about 1 tesla, or at least about 2 tesla, or at least about 3 tesla, or at least about 4 tesla, or at least about 5 tesla. In certain embodiments the magnetic stimulation is at a frequency of at least about 0.5 Hz, 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

Accordingly, in certain embodiments, methods of facilitating voiding of the bladder or bowel are provided where the methods involve providing magnetic stimulation of the spinal cord at a location, frequency and intensity sufficient to facilitate voiding of the bladder and/or bowel. In certain embodiments the spinal cord stimulation facilitates initiation of voiding of the bowel and/or bladder. In certain embodiments the spinal cord stimulation improves the efficacy of voiding of the bladder and/or bowel. In certain embodiments the spinal cord stimulation suppresses micturition. Also, in certain embodiments the magnetic stimulation is of a frequency and magnitude sufficient to restore volitional control of the bladder in the absence of stimulation.

Similarly, it was also observed that transcutaneous electrical stimulation can facilitate bladder and/or bowel control (see, e.g. Example 2). Transcutaneous electrical stimulation can readily be applied using an electrical stimulator coupled to electrodes that are applied to the surface of the subjects body (e.g., over the spinal cord at the regions described herein).

Suitable parameters for electrical stimulation and locations of such stimulation are discussed below and illustrated in Example 2.

### Regions of Stimulation

As noted above, in various embodiments one or more regions of the spinal cord are stimulated to facilitate locomotor function (e.g., standing, stepping, postural changes, arm and/or hand control, etc.), or to facilitate voiding of bowel and/or bladder. Depending on the desired function, in certain embodiments stimulation is applied to, or over, one or more regions of cervical spinal cord, and/or to or over one or more regions of the thoracic spinal cord, and/or to or over or one or more regions of the lumbosacral spinal cord.

For example, in certain embodiments, to facilitate locomotor activity such as standing, stepping, postural control, and the like, the methods may involve stimulating one or more regions of the thoracic and/or lumbosacral spinal cord.

In certain embodiments to facilitate locomotor activity such as control of the hand and/or arm and/or grasping, and the like, the methods may involve stimulating one or more regions of the cervical and/or thoracic spinal cord. Thus, for example, as demonstrated herein cervical spinal cord stimulation improves hand strength and hand and arm locomotor control.

In certain embodiments, to facilitate voiding of the bowel and/or bladder, the methods may involve stimulating one or more regions of the thoracic and/or lumbosacral spinal cord. For example, in certain embodiments, stimulation (e.g., magnetic stimulation) may be applied to or over one or more regions selected from the group consisting of T11-T12, L1-L2, and L2-L3. In certain embodiments stimulation (e.g., magnetic stimulation) may be applied to or over L1-L2 and/or T11-T12.

With respect to application of stimulation to the cervical spinal cord, illustrative regions include, but are not limited to one or more regions straddling or spanning a region selected from the group consisting of C1-C1, C1-C2, C1-C3, C1-C4, C1-C7, C1-C6, C1-C7, C1-T1, C2-C2, C2-C3, C2-C4, C2-C5, C2-C6, C2-C7, C2-T1, C3-C3, C3-C4, C3-C5, C3-C6, C3-C7, C3-T1, C4-C4, C4-C5, C4-C6, C4-C7, C4-T1, C5-C5, C5-C6, C5-C7, C5-T1, C6-C6, C6-C7, C6-T1, C7-C7, and C7-T1.

With respect to application of stimulation to the thoracic spinal cord, illustrative regions include, but are not limited to one or more regions straddling or spanning a region selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, and T12-T12.

With respect to application of stimulation to the lumbosacral spinal cord, illustrative regions include, but are not limited to one or more regions straddling or spanning a region selected from the group consisting of L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6.

### Methods of Stimulation

### Magnetic Stimulation

In certain embodiments the methods described herein utilize magnetic stimulators for stimulation of the spinal cord (e.g., spinal circuits) to facilitate locomotor activity (e.g., standing, stepping, sitting, laying down, stabilizing sitting posture, stabilizing standing posture, arm motion, hand motion, griping, hand strength, and the like) and/or to induce or improve voiding of the bowel and/or bladder. Magnetic spinal cord stimulation is achieved by generating a rapidly changing magnetic field to induce a current at the region(s) of interest. In certain embodiments effective spinal cord stimulation typically utilizes a current transient of about 10⁸ A/s or greater discharged through a stimulating coil. The discharge current flowing through the stimulating coil generates magnetic lines of force. As the lines of force cut through tissue (e.g., the spinal cord or brain stem), a current is generated in that tissue. If the induced current is of sufficient amplitude and duration such that the cell membrane is depolarized, neural/neuromuscular tissue will be stimulated.

Since the magnetic field strength falls off with the square of the distance from the stimulating coil, the stimulus strength is at its highest close to the coil surface. The stimulation characteristics of the magnetic pulse, such as depth of penetration, strength and accuracy, depend on the rise time, peak electrical energy transferred to the coil and the spatial distribution of the field. The rise time and peak coil energy are governed by the electrical characteristics of the magnetic stimulator and stimulating coil, whereas the spatial distribution of the induced electric field depends on the coil geometry and the anatomy of the region of induced current flow.

In various embodiments the magnetic nerve stimulator will produce a field strength up to about 10 tesla, or up to about 8 tesla, or up to about 6 tesla, or up to about 5 tesla, or up to about 4 tesla, or up to about 3 tesla, or up to about 2 tesla, or up to about 1 tesla, or up to about 0.8 tesla, or up to about 0.6 tesla, or up to about 0.5 tesla. In certain embodiments the nerve stimulator produces pulses with a duration from about 5 μs, or from about 10 μs, or from about 15 μs, or from about 20 μs up to about 10 ms, or from about 25 μs up to about 500 μs, or from about 25 μs or to about 100 μs, or from about 100 μs up to about 1 ms.

In certain embodiments the magnetic stimulation is at a frequency of at least about 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

In certain embodiments the magnetic stimulation is at a frequency ranging from about 0.5 Hz, or from about 1 Hz, or from about 2 Hz, or from about 3 Hz, or from about 4 Hz, or from about 5 Hz, or from about 10 Hz, or from about 10 Hz, or from about 10 Hz, up to about 500 Hz, or up to about 400 Hz, or up to about 300 Hz, or up to about 200 Hz up to about 100 Hz, or up to about 90 Hz, or up to about 80 Hz, or up to about 60 Hz, or up to about 40 Hz, or from about 3 Hz or from about 5 Hz up to about 80 Hz, or from about 5 Hz to about 60 Hz, or up to about 30 Hz.

In certain embodiments the magnetic stimulation is at a frequency ranging from about 20 Hz or about 30 Hz to about 90 Hz or to about 100 Hz.

In certain embodiments the magnetic stimulation is at a frequency, pulse width, and amplitude sufficient to initiate and/or improve standing, stepping, sitting, laying down, stabilizing sitting posture, stabilizing standing posture, arm motion, hand motion, stimulate gripping, improve hand strength, and the like, and/or to induce or improve voiding of the bowel and/or bladder. In certain embodiments the stimulation is at a frequency, pulse width, and amplitude sufficient to provide at least 30% emptying or at least 40% emptying, or at least 50% emptying, or at least 60% emptying, or at least 70% emptying, or at least 80% emptying, or at least 90% emptying, or at least 95% emptying, or at least 98% emptying of the bladder and/or bowel e.g., upon application of electrical stimulation as described herein.

### Transcutaneous Electrical Stimulation

In certain embodiments the methods described herein utilize transcutaneous electrical stimulation for stimulation of the spinal cord (e.g., spinal circuits) to facilitate locomotor activity (e.g., standing, stepping, sitting, laying down, stabilizing sitting posture, stabilizing standing posture, arm motion, hand motion, griping, hand strength, and the like) and/or to induce or improve voiding of the bowel and/or bladder. The use of surface electrode(s), can facilitates selection or alteration of particular stimulation sites as well as the application of a wide variety of stimulation parameters. Additionally surface stimulation can be used to optimize location for an implantable electrode or electrode array for epidural stimulation.

In various embodiments, the methods described herein involve transcutaneous electrical stimulation of the cervical spine or a region of the cervical spine and/or the thoracic spinal cord or a region of the thoracic spinal cord, and/or a region of the lumbosacral spinal cord as described herein to facilitate locomotor activity and/or voiding of the bowel and/or bladder (e.g., as described above).

In certain embodiments the transcutaneous stimulation is at a frequency of at least about 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

In certain embodiments the transcutaneous stimulation is at a frequency ranging from about 1 Hz, or from about 2 Hz, or from about 3 Hz, or from about 4 Hz, or from about 5 Hz, or from about 10 Hz, or from about 10 Hz, or from about 10 Hz, up to about 500 Hz, or up to about 400 Hz, or up to about 300 Hz, or up to about 200 Hz up to about 100 Hz, or up to about 90 Hz, or up to about 80 Hz, or up to about 60 Hz, or up to about 40 Hz, or from about 3 Hz or from about 5 Hz up to about 80 Hz, or from about 5 Hz to about 60 Hz, or up to about 30 Hz. In certain embodiments the transcutaneous stimulation is at a frequency ranging from about 20 Hz or about 30 Hz to about 90 Hz or to about 100 Hz.

In certain embodiments the transcutaneous stimulation is applied at an intensity ranging from about 5 mA or about 10 mA up to about 500 mA, or from about 5 mA or about 10 mA up to about 400 mA, or from about 5 mA or about 10 mA up to about 300 mA, or from about 5 mA or about 10 mA up to about 200 mA, or from about 5 mA or about 10 mA to up about 150 mA, or from about 5 mA or about 10 mA up to about 50 mA, or from about 5 mA or about 10 mA up to about 100 mA, or from about 5 mA or about 10 mA up to about 80 mA, or from about 5 mA or about 10 mA up to about 60 mA, or from about 5 mA or about 10 mA up to about 50 mA.

In certain embodiments the transcutaneous stimulation is applied stimulation comprises pulses having a width that ranges from about 100 μs up to about 1 ms or up to about 800 μs, or up to about 600 μs, or up to about 500 μs, or up to about 400 μs, or up to about 300 μs, or up to about 200 μs, or up to about 100 μs, or from about 150 μs up to about 600 μs, or from about 200 μs up to about 500 μs, or from about 200 μs up to about 400 μs.

In certain embodiments the transcutaneous stimulation is at a frequency, pulse width, and amplitude sufficient to initiate and/or improve standing, stepping, sitting, laying down, stabilizing sitting posture, stabilizing standing posture, arm motion, hand motion, griping, hand strength, and the like) and/or to induce or improve voiding of the bowel and/or bladder. In certain embodiments the stimulation is at a frequency, pulse width, and amplitude sufficient to provide at least 30% emptying or at least 40% emptying, or at least 50% emptying, or at least 60% emptying, or at least 70% emptying, or at least 80% emptying, or at least 90% emptying, or at least 95% emptying, or at least 98% emptying of the bladder and/or bowel e.g., upon application of electrical stimulation as described herein.

In certain embodiments the transcutaneous stimulation is superimposed on a high frequency carrier signal. In certain embodiments the high frequency carrier signal ranges from about 3 kHz, or about 5 kHz, or about 8 kHz up to about 30 kHz, or up to about 20 kHz, or up to about 15 kHz. In certain embodiments the carrier signal is about 10 kHz. In certain embodiments the carrier frequency amplitude ranges from about 30 mA, or about 40 mA, or about 50 mA, or about 60 mA, or about 70 mA, or about 80 mA up to about 300 mA, or up to about 200 mA, or up to about 150 mA.

Accordingly, in certain embodiments, the transcutaneous stimulation is applied as a high frequency signal that is pulsed at a frequency ranging from about 1 Hz up to about 100 Hz as described above. In one illustrative but non-limiting embodiment, the stimulation is a 1 Hz transcutaneous electrical stimulation evoked with a 10 kHz constant-current bipolar rectangular stimulus for 0.5 ms at 30 to 100 mA repeated at 1-40 times per second for 10 to 30 s. This results in a low (2% or less) duty cycle that is well tolerated. In certain embodiments the voltage is approximately 30 V at 100 mA. In certain embodiments each stimulation epoch is repeated 1-10, or 1-5 times per session, once per week for, e.g., 6-12 weeks.

### Epidural Stimulation

In various embodiments, the methods described herein can involve epidural electrical stimulation for stimulation of the spinal cord (e.g., spinal circuits) to facilitate locomotor activity (e.g., standing, stepping, sitting, laying down, stabilizing sitting posture, stabilizing standing posture, arm motion, hand motion, griping, hand strength, and the like) and/or to induce or improve voiding of the bowel and/or bladder.

In certain embodiments, the epidural stimulation is at a frequency of at least about 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

In certain embodiments, the epidural stimulation is at a frequency ranging from about 1 Hz, or from about 2 Hz, or from about 3 Hz, or from about 4 Hz, or from about 5 Hz, or from about 10 Hz, or from about 10 Hz, or from about 10 Hz, up to about 500 Hz, or up to about 400 Hz, or up to about 300 Hz, or up to about 200 Hz up to about 100 Hz, or up to about 90 Hz, or up to about 80 Hz, or up to about 60 Hz, or up to about 40 Hz, or from about 3 Hz or from about 5 Hz up to about 80 Hz, or from about 5 Hz to about 60 Hz, or up to about 30 Hz.

In certain embodiments, the epidural stimulation is at a frequency ranging from about 20 Hz or about 30 Hz to about 90 Hz or to about 100 Hz.

In certain embodiments the epidural stimulation is at a frequency, pulse width, and amplitude sufficient to initiate and/or improve standing, stepping, sitting, laying down, stabilizing sitting posture, stabilizing standing posture, arm motion, hand motion, stimulate gripping, improve hand strength, and the like, and/or to induce or improve voiding of the bowel and/or bladder. In certain embodiments the stimulation is at a frequency, pulse width, and amplitude sufficient to provide at least 30% emptying or at least 40% emptying, or at least 50% emptying, or at least 60% emptying, or at least 70% emptying, or at least 80% emptying, or at least 90% emptying, or at least 95% emptying, or at least 98% emptying of the bladder and/or bowel e.g., upon application of electrical stimulation as described herein.

In certain embodiments, the epidural stimulation is at an amplitude ranging from 0.5 mA, or from about 1 mA, or from about 2 mA, or from about 3 mA, or from about 4 mA, or from about 5 mA up to about 50 mA, or up to about 30 mA, or up to about 20 mA, or up to about 15 mA, or from about 5 mA to about 20 mA, or from about 5 mA up to about 15 mA.

In certain embodiments, the epidural stimulation is with pulses having a pulse width ranging from about 100 μs up to about 1 ms or up to about 800 μs, or up to about 600 μs, or up to about 500 μs, or up to about 400 μs, or up to about 300 μs, or up to about 200 μs, or up to about 100 μs, or from about 150 μs up to about 600 μs, or from about 200 μs up to about 500 μs, or from about 200 μs up to about 400 μs.

In certain embodiments the epidural stimulation is applied paraspinally over a cervical region identified above (e.g., over vertebrae spanning C0 to C8 or a region thereof, e.g., over a region spanning C3 to C4).

In certain embodiments, the epidural stimulation is applied via a permanently implanted electrode array (e.g., a typical density electrode array, a high density electrode array, etc.).

In certain embodiments, the epidural electrical stimulation is administered via a high density epidural stimulating array (e.g., as described in PCT Publication No: WO/2012/094346 (PCT/US2012/020112). In certain embodiments, the high density electrode arrays are prepared using microfabrication technology to place numerous electrodes in an array configuration on a flexible substrate. In some embodiments, epidural array fabrication methods for retinal stimulating arrays can be used in the methods described herein (see, e.g., Maynard (2001) Annu. Rev. Biomed. Eng., 3: 145-168; Weiland and Humayun (2005) IEEE Eng. Med. Biol. Mag., 24(5): 14-21, and U.S. Patent Publications 2006/0003090 and 2007/0142878). In various embodiments, the stimulating arrays comprise one or more biocompatible metals (e.g., gold, platinum, chromium, titanium, iridium, tungsten, and/or oxides and/or alloys thereof) disposed on a flexible material. Flexible materials can be selected from parylene A, parylene C, parylene AM, parylene F, parylene N, parylene D, other flexible substrate materials, or combinations thereof. Parylene has the lowest water permeability of available microfabrication polymers, is deposited in a uniquely conformal and uniform manner, has previously been classified by the FDA as a United States Pharmacopeia (USP) Class VI biocompatible material (enabling its use in chronic implants) (Wolgemuth, Medical Device and Diagnostic Industry, 22(8): 42-49 (2000)), and has flexibility characteristics (Young's modulus ˜4 GPa (Rodger and Tai (2005) IEEE Eng. Med. Biology, 24(5): 52-57)), lying in between those of PDMS (often considered too flexible) and most polyimides (often considered too stiff). Finally, the tear resistance and elongation at break of parylene are both large, minimizing damage to electrode arrays under surgical manipulation. The preparation and parylene microelectrode arrays suitable for use in the epidural stimulation methods described herein is described in PCT Publication No: WO/2012/100260 (PCT/US2012/022257).

The electrode array may be implanted using any of a number of methods (e.g., a laminectomy procedure) well known to those of skill in the art. For example, in some embodiments, electrical energy is delivered through electrodes positioned external to the dura layer surrounding the spinal cord. Stimulation on the surface of the cord (subdurally) is also contemplated, for example, stimulation may be applied to the dorsal columns as well as to the dorsal root entry zone. In certain embodiments the electrodes are carried by two primary vehicles: a percutaneous lead and a laminotomy lead. Percutaneous leads can typically comprise two or more, spaced electrodes (e.g., equally spaced electrodes), that are placed above the dura layer, e.g., through the use of a Touhy-like needle. For insertion, the Touhy-like needle can be passed through the skin, between desired vertebrae, to open above the dura layer. An example of an eight-electrode percutaneous lead is an OCTRODE® lead manufactured by Advanced Neuromodulation Systems, Inc.

Laminotomy leads typically have a paddle configuration and typically possess a plurality of electrodes (for example, two, four, eight, sixteen. 24, or 32) arranged in one or more columns. An example of an eight-electrode, two column laminotomy lead is a LAMITRODE® 44 lead manufactured by Advanced Neuromodulation Systems, Inc. In certain embodiments the implanted laminotomy leads are transversely centered over the physiological midline of a subject. In such position, multiple columns of electrodes are well suited to administer electrical energy on either side of the midline to create an electric field that traverses the midline. A multi-column laminotomy lead enables reliable positioning of a plurality of electrodes, and in particular, a plurality of electrode rows that do not readily deviate from an initial implantation position.

Laminotomy are typically implanted in a surgical procedure. The surgical procedure, or partial laminectomy, typically involves the resection and removal of certain vertebral tissue to allow both access to the dura and proper positioning of a laminotomy lead. The laminotomy lead offers a stable platform that is further capable of being sutured in place.

In the context of conventional spinal cord stimulation, the surgical procedure, or partial laminectomy, typically involves the resection and removal of certain vertebral tissue to allow both access to the dura and proper positioning of a laminotomy lead. Depending on the position of insertion, however, access to the dura may only require a partial removal of the ligamentum flavum at the insertion site. In certain embodiments, two or more laminotomy leads are positioned within the epidural space of C1-C7 as identified above. The leads may assume any relative position to one another.

In various embodiments, the arrays are operably linked to control circuitry that permits selection of electrode(s) to activate/stimulate and/or that controls frequency, and/or pulse width, and/or amplitude of stimulation. In various embodiments, the electrode selection, frequency, amplitude, and pulse width are independently selectable, e.g., at different times, different electrodes can be selected. At any time, different electrodes can provide different stimulation frequencies and/or amplitudes. In various embodiments, different electrodes or all electrodes can be operated in a monopolar mode and/or a bipolar mode, using constant current or constant voltage delivery of the stimulation. In certain embodiments time-varying current and/or time-varying voltage may be utilized.

In certain embodiments, the electrodes can also be provided with implantable control circuitry and/or an implantable power source. In various embodiments, the implantable control circuitry can be programmed/reprogrammed by use of an external device (e.g., using a handheld device that communicates with the control circuitry through the skin). The programming can be repeated as often as necessary.

The epidural electrode stimulation systems described herein are intended to be illustrative and non-limiting. Using the teachings provided herein, alternative epidural stimulation systems and methods will be available to one of skill in the art.

### Stimulators and Stimulation Systems

### Magnetic Stimulators

Magnetic nerve stimulators are well known to those of skill in the art. Stimulation is achieved by generating a rapidly changing magnetic field to induce a current at the nerve of interest. Effective nerve stimulation typically requires a current transient of about 10⁸ A/s. In certain embodiments this current is obtained by switching the current through an electronic switching component (e.g., a thyristor or an insulated gate bipolar transistor (IGBT)).

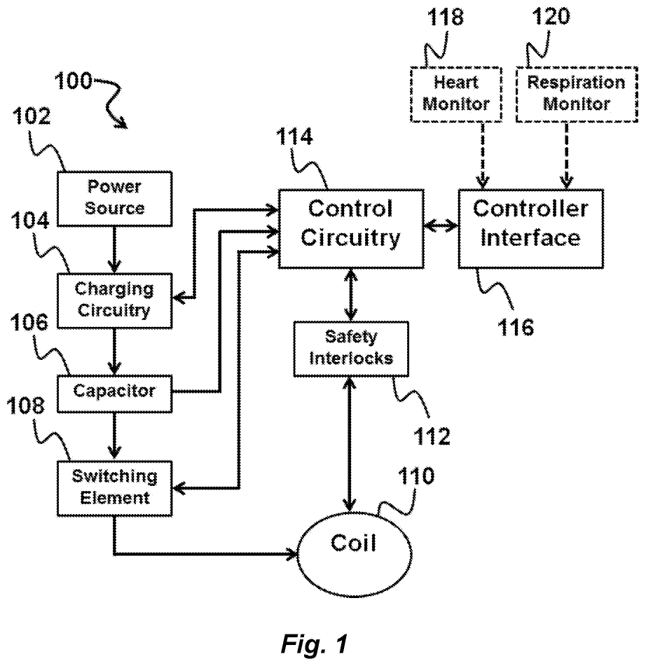

FIG. 1 schematically shows one illustrative, but non-limiting embodiment of a magnetic stimulator. As shown therein, magnetic nerve stimulator 100 comprises two parts: a high current pulse generator producing discharge currents of, e.g., 5,000 amps or more; and a stimulating coil 110 producing magnetic pulses (e.g., with field strengths up to 4, 6, 8, or even 10 tesla) and with a pulse duration typically ranging from about 100 μs to 1 ms or more, depending on the stimulator type. As illustrated in FIG. 1, a voltage (power) source 102 (e.g., a battery) charges a capacitor 106 via charging circuitry 104 under the control of control circuitry 114 (e.g., a microprocessor) that accepts information such as the capacitor voltage, power set by the user, and various safety interlocks 112 within the equipment to ensure proper operation, and the capacitor is then connected to the coil via an electronic switching component 108 when the stimulus is to be applied. The control circuitry is operated via a controller interface 116 that can receive user input and/optionally signals from external sources such as internet monitors, health care professionals, and the like.

When activated, the discharge current flows through the coils inducing a magnetic flux. It is the rate of change of the magnetic field that causes the electrical current within tissue to be generated, and therefore a fast discharge time is important to stimulator efficiency.

As noted earlier the magnetic field is simply the means by which an electrical current is generated within the tissue, and that it is the electrical current, and not the magnetic field, that causes the depolarization of the cell membrane and thus the stimulation of the target nerve.

Since the magnetic field strength falls off with the square of the distance from the stimulating coil, the stimulus strength is at its highest close to the coil surface. The stimulation characteristics of the magnetic pulse, such as depth of penetration, strength and accuracy, depend on the rise time, peak electrical energy transferred to the coil and the spatial distribution of the field. The rise time and peak coil energy are governed by the electrical characteristics of the magnetic stimulator and stimulating coil, whereas the spatial distribution of the induced electric field depends on the coil geometry and the anatomy of the region of induced current flow.

The stimulating coils typically consist of one or more well-insulated copper windings, together with temperature sensors and safety switches.

In certain embodiments the use of single coils is contemplated. Single coils are effective in stimulating the human motor cortex and spinal nerve roots. To date, circular coils with a mean diameter of 80-100 mm have remained the most widely used magnetic stimulation. In the case of circular coils the induced tissue current is near z on the central axis of the coil and increases to a maximum in a ring under the mean diameter of coil.

A notable improvement in coil design has been that of the double coil (also termed butterfly or figure eight coil). Double coils utilize two windings, normally placed side by side. Typically double coils range from very small flat coils to large contoured versions. The main advantage of double coils over circular coils is that the induced tissue current is at its maximum directly under the center where the two windings meet, giving a more accurately defined area of stimulation. In certain embodiments, the use of an angled butterfly coil may provide improved effects of stimulation.

The stimulating pulse may be monophasic, symmetrical biphasic (with or without an interphase gap), asymmetric biphasic (with or without an interphase gal), or symmetric or asymmetric polyphasic (e.g., burst stimulation having a particular burst duration and carrier frequency). Each of these has its own properties and so may be useful in particular circumstances. For neurology, single pulse, monophasic systems are generally employed; for rapid rate stimulators, biphasic systems are used as energy must be recovered from each pulse in order to help fund the next. Polyphasic stimulators are believed to have a role in a number of therapeutic applications.

Descriptions of magnetic nerve stimulators can be found, inter alia, in U.S. patent publications US 2009/0108969 A1, US 2013/0131753 A1, US 2012/0101326 A1, IN U.S. Pat. Nos. 8,172,742, 6,086,525, 5,066,272, 6,500,110, 8,676,324, and the like. Magnetic stimulators are also commercially availed from a number of vendors, e.g., MAGVENTURE®, MAGSTIM®, and the like.

### Electrical Stimulators

Any present or future developed stimulation system capable of providing an electrical signal to one or more regions of the cervical spinal cord may be used in accordance with the teachings provided herein. Electrical stimulation systems (e.g., pulse generator(s)) can be used with both transcutaneous stimulation and epidural stimulation.

In various embodiments, the system may comprise an external pulse generator for use with either a transcutaneous stimulation system or an epidural system. In other embodiments the system may comprise an implantable pulse generator to produce a number of stimulation pulses that are sent to the a region in proximity to the cervical spinal cord by insulated leads coupled to the spinal cord by one or more electrodes and/or an electrode array to provide epidural stimulation. In certain embodiments the one or more electrodes or one or more electrodes comprising the electrode array may be attached to separate conductors included within a single lead. Any known or future developed lead useful for applying an electrical stimulation signal in proximity to a subject's spinal cord may be used. For example, the leads may be conventional percutaneous leads, such as PISCES® model 3487A sold by Medtronic, Inc. In some embodiments, it may be desirable to employ a paddle-type lead.

Any known or future developed external or implantable pulse generator may be used in accordance with the teachings provided herein. For example, one internal pulse generator may be an ITREL® II or Synergy pulse generator available from Medtronic, Inc, Advanced Neuromodulation Systems, Inc.'s GENESIS™ pulse generator, or Advanced Bionics Corporation's PRECISION™ pulse generator. One of skill in the art will recognize that the above-mentioned pulse generators may be advantageously modified to modulate locomotor function and/or bladder and/or bowel control in accordance with the teachings provided herein.

In certain embodiments systems can employ a programmer coupled via a conductor to a radio frequency antenna. This system permits attending medical personnel to select the various pulse output options after implant using radio frequency communications. While, in certain embodiments, the system employs fully implanted elements, systems employing partially implanted elements may also be used in accordance with the teachings provided herein.

In one illustrative, but non-limiting system, a control module is operably coupled to a signal generation module and instructs the signal generation module regarding the signal to be generated. For example, at any given time or period of time, the control module may instruct the signal generation module to generate an electrical signal having a specified pulse width, frequency, intensity (current or voltage), etc. The control module may be preprogrammed prior to implantation or receive instructions from a programmer (or another source) through any known or future developed mechanism, such as telemetry. The control module may include or be operably coupled to memory to store instructions for controlling the signal generation module and may contain a processor for controlling which instructions to send to signal generation module and the timing of the instructions to be sent to signal generation module.

In certain embodiments, the controller alters and/or locomotor function and/or initiates or facilitates voiding of the bladder and/or bowel on demand.

In various embodiments, leads are operably coupled to signal generation module such that a stimulation pulse generated by signal generation module may be delivered via electrodes.

While in certain embodiments, two leads are utilized, it will be understood that any number of one or more leads may be employed. In addition, it will be understood that any number of one or more electrodes per lead may be employed. Stimulation pulses are applied to electrodes (which typically are cathodes) with respect to a return electrode (which typically is an anode) to induce a desired area of excitation of electrically excitable tissue in a region of the cervical spine. A return electrode such as a ground or other reference electrode can be located on same lead as a stimulation electrode. However, it will be understood that a return electrode may be located at nearly any location, whether in proximity to the stimulation electrode or at a more remote part of the body, such as at a metallic case of a pulse generator. It will be further understood that any number of one or more return electrodes may be employed. For example, there can be a respective return electrode for each cathode such that a distinct cathode/anode pair is formed for each cathode.

In various embodiments, the independent electrodes or electrodes of electrode arrays are operably linked to control circuitry that permits selection of electrode(s) to activate/stimulate and/or controls frequency, and/or pulse width, and/or amplitude of stimulation. In various embodiments, the electrode selection, frequency, amplitude, and pulse width are independently selectable, e.g., at different times, different electrodes can be selected. At any time, different electrodes can provide different stimulation frequencies and/or amplitudes. In various embodiments, different electrodes or all electrodes can be operated in a monopolar mode and/or a bipolar mode, using, e.g., constant current or constant voltage delivery of the stimulation.

In one illustrative but non-limiting system a control module is operably coupled to a signal generation module and instructs the signal generation module regarding the signal to be generated. For example, at any given time or period of time, the control module may instruct the signal generation module to generate an electrical signal having a specified pulse width, frequency, intensity (current or voltage), etc. The control module may be preprogrammed prior to use or receive instructions from a programmer (or another source). Thus, in certain embodiments the pulse generator/controller is configurable by software and the control parameters may be programmed/entered locally, or downloaded as appropriate/necessary from a remote site.

In certain embodiments the pulse generator/controller may include or be operably coupled to memory to store instructions for controlling the stimulation signal(s) and may contain a processor for controlling which instructions to send for signal generation and the timing of the instructions to be sent.

While in certain embodiments, two leads are utilized to provide transcutaneous or epidural stimulation, it will be understood that any number of one or more leads may be employed. In addition, it will be understood that any number of one or more electrodes per lead may be employed. Stimulation pulses are applied to electrodes (which typically are cathodes) with respect to a return electrode (which typically is an anode) to induce a desired area of excitation of electrically excitable tissue in one or more regions of the spine. A return electrode such as a ground or other reference electrode can be located on same lead as a stimulation electrode. However, it will be understood that a return electrode may be located at nearly any location, whether in proximity to the stimulation electrode or at a more remote part of the body, such as at a metallic case of a pulse generator. It will be further understood that any number of one or more return electrodes may be employed. For example, there can be a respective return electrode for each cathode such that a distinct cathode/anode pair is formed for each cathode.

### Use of Neuromodulatory Agents

In certain embodiments, the transcutaneous and/or epidural and/or magnetic stimulation methods described herein are used in conjunction with various pharmacological agents, particularly pharmacological agents that have neuromodulatory activity (e.g., are monoamergic). In certain embodiments, the use of various serotonergic, and/or dopaminergic, and/or noradrenergic, and/or GABAergic, and/or glycinergic drugs is contemplated. These agents can be used in conjunction with epidural stimulation and/or transcutaneous stimulation and/or magnetic stimulation as described above. This combined approach can help to put the spinal cord in an optimal physiological state for neuromodulation utilizing the methods described herein.

In certain embodiments, the drugs are administered systemically, while in other embodiments, the drugs are administered locally, e.g., to particular regions of the spinal cord. Drugs that modulate the excitability of the spinal neuromotor networks include, but are not limited to combinations of noradrenergic, serotonergic, GABAergic, and glycinergic receptor agonists and antagonists.

Dosages of at least one drug or agent can be between about 0.001 mg/kg and about 10 mg/kg, between about 0.01 mg/kg and about 10 mg/kg, between about 0.01 mg/kg and about 1 mg/kg, between about 0.1 mg/kg and about 10 mg/kg, between about 5 mg/kg and about 10 mg/kg, between about 0.01 mg/kg and about 5 mg/kg, between about 0.001 mg/kg and about 5 mg/kg, or between about 0.05 mg/kg and about 10 mg/kg.

Drugs or agents can be delivery by injection (e.g., subcutaneously, intravenously, intramuscularly), orally, rectally, or inhaled.

Illustrative pharmacological agents include, but are not limited to, agonists and antagonists to one or more combinations of serotonergic: 5-HT1A, 5-HT2A, 5-HT3, and 5HT7 receptors; to noradrenergic alpha 1 and 2 receptors; and to dopaminergic D1 and D2 receptors (see, e.g., Table 1). In certain embodiments, suitable pharmacological agents may include selective serotonin reuptake inhibitors (SSRI) such as fluoxetine, etc.

TABLE 1
Illustrative pharmacological agents.

| | | | | | |
|---|---|---|---|---|---|
| | | | | Typical | Typical |
| | | | | Dose | Range |
| Name | Target | Action | Route | (mg/Kg) | (mg/kg) |

Serotonergic receptor systems

| | | | | | |
|---|---|---|---|---|---|
| 8-OHDPAT | 5-HT1A7 | Agonist | S.C. | 0.05 | 0.045-0.3 |
| Way 100.635 | 5-HT1A | Antagonist | I.P. | 0.5 | 0.4-1.5 |
| Quipazine | 5-HT2A/C | Agonist | I.P. | 0.2 | 0.18-0.6 |
| Ketanserin | 5-HT2A/C | Antagonist | I.P. | 3 | 1.5-6.0 |
| SR 57227A | 5-HT3 | Agonist | I.P. | 1.5 | 1.3-1.7 |
| Ondanesetron | 5-HT3 | Antagonist | I.P. | 3 | 1.4-7.0 |
| SB269970 | 5-HT7 | Antagonist | I.P. | 7 | 2.0-10.0 |

Noradrenergic receptor systems

| | | | | | |
|---|---|---|---|---|---|
| Methoxamine | Alpha1 | Agonist | I.P. | 2.5 | 1.5-4.5 |
| Prazosin | Alpha1 | Antagonist | I.P. | 3 | 1.8-3.0 |
| Clonidine | Alpha2 | Agonist | I.P. | 0.5 | 0.2-1.5 |
| Yohimbine | Alpha2 | Antagonist | I.P. | 0.4 | 0.3-0.6 |

Dopaminergic receptor systems

| | | | | | |
|---|---|---|---|---|---|
| SKF-81297 | D1-like | Agonist | I.P. | 0.2 | 0.15-0.6 |
| SCH-23390 | D1-like | Antagonist | I.P. | 0.15 | 0.1-0.75 |
| Quinipirole | D2-like | Agonist | I.P. | 0.3 | 0.15-0.3 |
| Eticlopride | D2-like | Antagonist | I.P. | 1.8 | 0.9-1.8 |

The foregoing methods are intended to be illustrative and non-limiting. Using the teachings provided herein, other methods involving transcutaneous electrical stimulation and/or epidural electrical stimulation and/or magnetic stimulation and/or the use of neuromodulatory agents to facilitate voiding or control of bladder and/or bowel in a subject with dysfunctional bladder and/or bowel function will be available to one of skill in the art.

### EXAMPLES

The following examples are offered to illustrate, but not to limit the claimed invention.

### Example 1

### A Proof-of-Concept Study of Transcutaneous Magnetic Spinal Cord Stimulation for Neurogenic Bladder

Patients with chronic spinal cord injury (SCI) cannot urinate at will and must empty the bladder by self-catheterization. We tested the hypothesis that non-invasive, transcutaneous magnetic spinal cord stimulation (TMSCS) would improve bladder function in individuals with SCI. Five individuals with American Spinal Injury Association Impairment Scale A/B, chronic SCI and detrusor sphincter dyssynergia enrolled in this prospective, interventional study.

After a two-week assessment to determine effective stimulation characteristics, each patient received sixteen weekly TMSCS treatments and then received “sham” weekly stimulation for six weeks while bladder function was monitored. Bladder function improved in all five subjects, but only during and after repeated weekly sessions of 1 Hz TMSCS. All subjects achieved volitional urination. The volume of urine produced voluntarily increased from 0 cc/day to 1120 cc/day (p=0.03); self-catheterization frequency decreased from 6.6/day to 2.4/day (p=0.04); the capacity of the bladder increased from 244 ml to 404 ml (p=0.02); and the average quality of life ranking increased significantly (p=0.007). Volitional bladder function was re-enabled in five individuals with SCI following intermittent, non-invasive TMSCS. We conclude that neuromodulation of spinal micturition circuitry by TMSCS may be used to ameliorate bladder function.

### Results

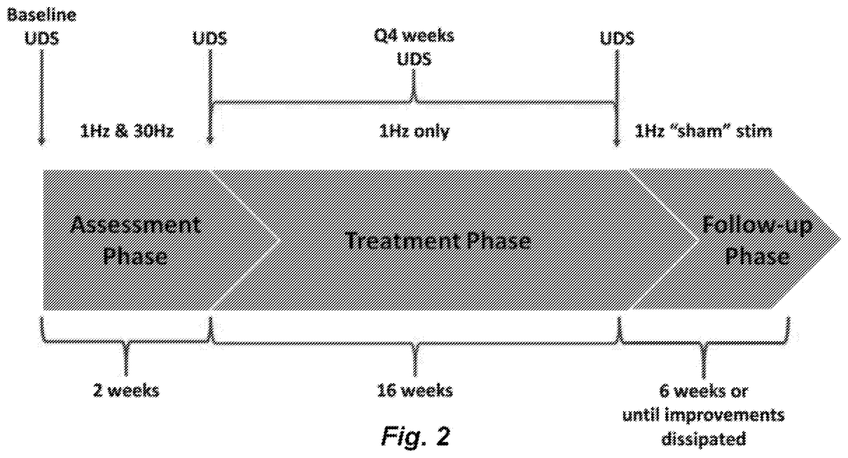

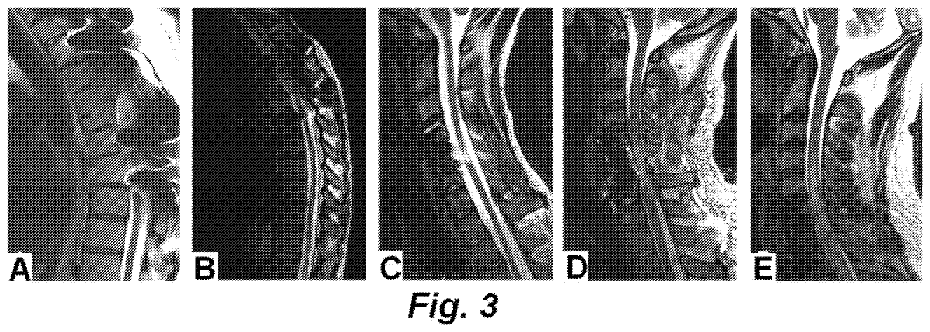

Subjects underwent 3 study phases as illustrated in FIG. 2. Demographic information and indices of bladder function for all five subjects are shown in Table 2. The magnetic resonance images (MRI) indicating the level and extent of SCI for each subject are shown in FIG. 3. The average duration of SCI was 8.8±7.5 years. None of the subjects had been able to void voluntarily since the time of injury as shown in at least three prior urodynamic studies in each subject.

TABLE 2
Demographic information, the origin and nature of the SCI and urinary indices.

| | | | | | | | Length of | Decal |
|---|---|---|---|---|---|---|---|---|
| | | | | | | | stimulation | of |
| | | | | | | | until | the |
| | | | | | Disease | Mechanism | volitional | effect |
| | | | Injury | ASIA | Duration | of | micturition | duration |
| # | Sex | Age | Level | Grade | (years) | Injury | (weeks) | (weeks) |
| A | M | 42 | T4 | A | 13 | MVA | 4 | 4 |
| B | M | 43 | T4 | A | 5 | Wrestling | 6 | 3 |
| C | M | 22 | C5 | B | 8 | Football | 5 | 3 |
| D | M | 25 | C6 | B | 8 | MVA | 5 | 4 |
| E | M | 23 | C7 | A | 8 | MVA | 8 | 2 |
| Avg | — | — | — | — | — | — | 5.6 | 3.2 |
| SD | — | — | — | — | — | — | 1.5 | 0.8 |

| | | | | | | | Daily |
|---|---|---|---|---|---|---|---|
| | CIC/ | CIC/ | Volitional | Stream | Bladder | Bladder | Voiding |
| | day | day | Void | Velocity | Capacity | Capacity | post |
| | Pre | Post | (Y/N) | (ml/s) | Pre (ml) | Post (ml) | (ml) |
| A | 9 | 0 | Y | 10 | 141 | 431 | 2000 |
| B | 6 | 3 | Y | 10 | 238 | 462 | 700 |
| C | 6 | 3 | Y | 10 | 270 | 351 | 800 |
| D | 6 | 1 | Y | 8 | 215 | 325 | 1800 |
| E | 6 | 5 | Y | 8 | 354 | 452 | 300 |
| Avg | 6.6 | 2.4 | — | 9.3 | 244 | 404 | 1120 |
| SD | 1.3 | 1.9 | — | 1.1 | 78 | 62 | 740 |

Ais = American Spinal Injury Association Impairment Scale.
MVA= motor vehicle accident.
CIC = clean intermittent catheterization.

### Determining the Optimal Frequency: Spinal Function

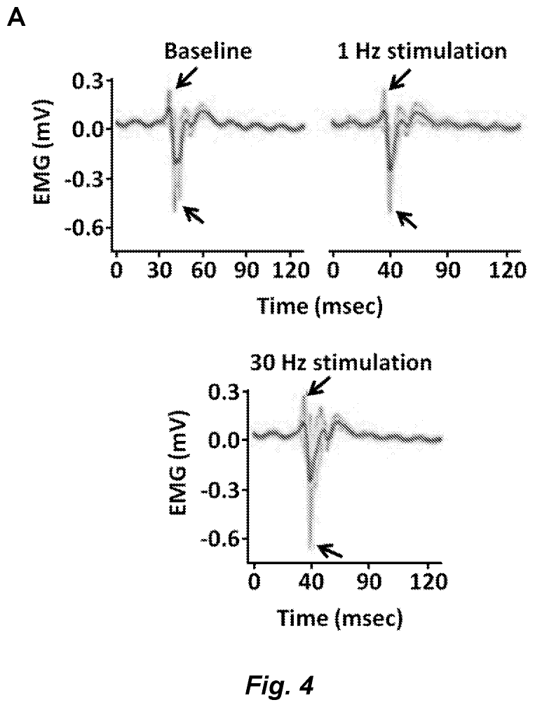

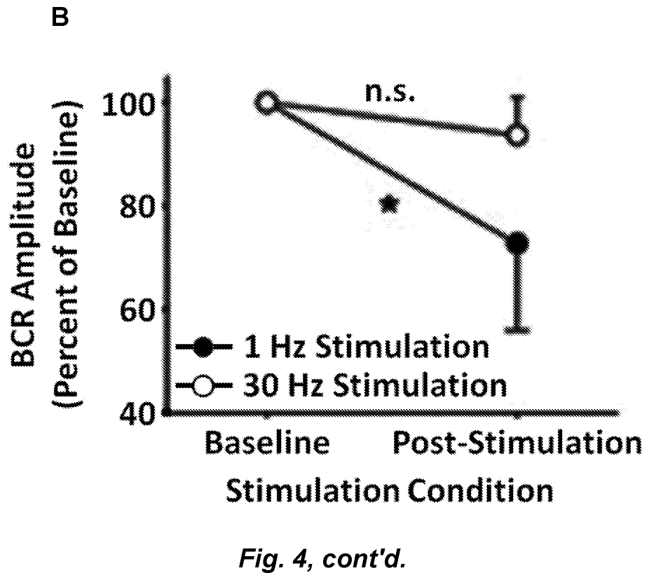

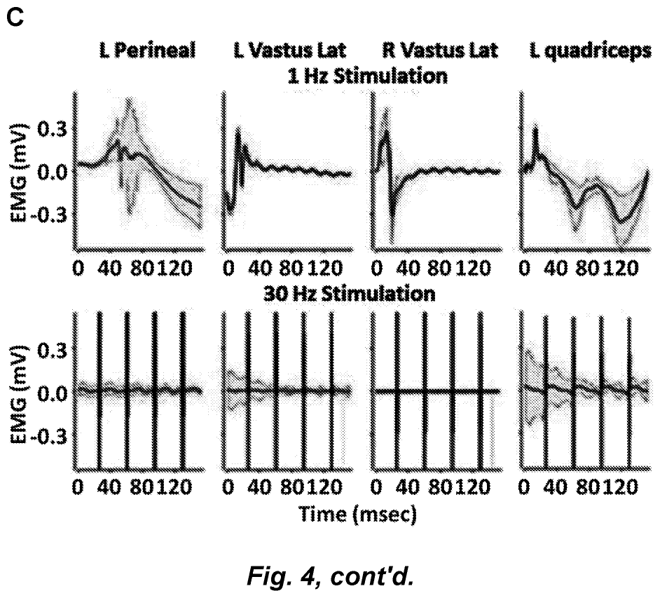

The bulbocavernosus reflex (BCR) is disinhibited and pathologically hyperactive after SCI (FIG. 4). The BCR amplitude was significantly reduced during 1 Hz TMSCS in all five subjects (p<0.001). In contrast, high frequency stimulation either increased the BCR amplitude or had no significant effect. The average BCR latency was 35.2±5.3 ms during both 1 Hz and 30 Hz TMSCS, which is similar to the latency of the BCR in normal individuals (Granata et al. (2013) Func. Neural., 28: 293-295).

During 1 Hz TMSCS, spinal cord evoked potentials could be elicited in selected lower extremity muscle groups (perineal, vastus lateralis and quadriceps femoris); whereas we were unable to detect any spinal evoked potentials at 30 Hz stimulation (FIG. 4)

### Determining the Optimal Frequency: Bladder Function

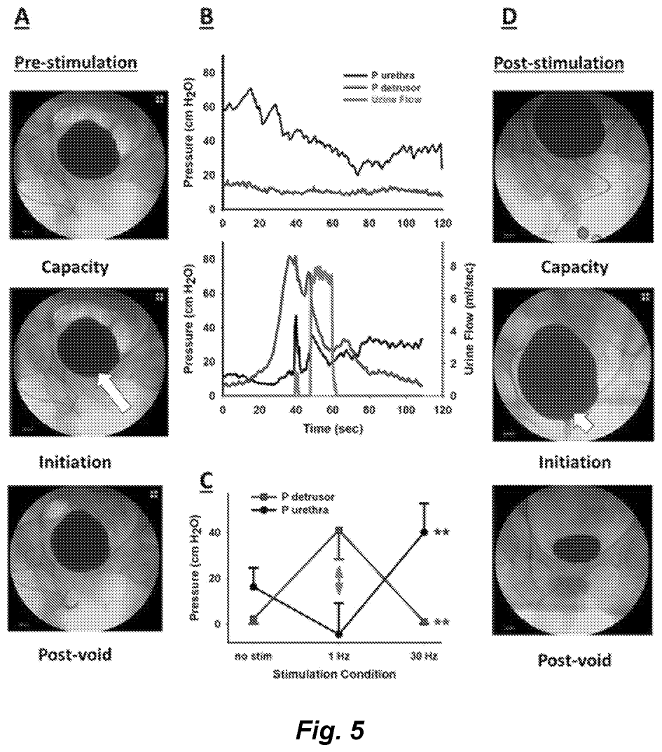

During the assessment phase, the urethral (P urethra) and detrusor pressures (P detrusor) obtained during urodynamic testing during attempted volitional micturition were significantly different during high and low frequency TMSCS (FIG. 5). The urethral and detrusor pressures are shown in Tables 3 and 4, respectively. The average urethral pressure was significantly lower than the baseline, unstimulated value during 1 Hz stimulation (p<0.05) and the average urethral pressure was greater than the unstimulated, baseline value during 30 Hz stimulation (though this was not statistically significant P<0.10). On the other hand, the average detrusor pressure was significantly elevated during 1 Hz stimulation compared to both the baseline, unstimulated condition and 30 Hz stimulation (P<0.01 for both comparisons), and the detrusor pressure was not different from baseline conditions during 30 Hz stimulation (p=0.5). Thus, stimulation at low frequency allowed each subject to elevate the bladder pressure and reduce the urethral pressure (conditions conducive to urine flow), and 30 Hz stimulation had the opposite effect: urethral pressure increased significantly, but detrusor pressure was not modified by 30 Hz stimulation (Tables 3 and 4). Not surprisingly, increasing detrusor contraction and bladder pressure while simultaneously decreasing urethral pressure allowed voluntary micturition (FIG. 5).

TABLE 3
At the end of the assessment phase, changes in urethral pressure in five
subjects during micturition attempts compared to the pre-attempt
baseline. Positive numbers indicate an increase in the pressure
during the attempts while negative numbers indicate a decrease in
the urethral pressure during the attempts. Notice that high
frequency stimulation (30 Hz) resulted in increased/unchanged
urethral pressure during attempted micturition when compared
to the no-stimulation/baseline; low frequency stimulation (1 Hz),
on the other hand, significant decreased urethral pressure compared
to both unstimulated and 30 Hz stimulation conditions.

| | No | | |
|---|---|---|---|
| Δ P ure | Stimulation | Low Frequency | High Frequency |
| (mmHg) | (NoS) | (1 Hz) | (30 Hz) |
| Subject A | 28.4 ± 5.0 | −25.3 ± 2.7 | 36.2 ± 11.1 |
| Subject B | 2.5 ± 4.8 | 0.4 ± 5.2 | 50.3 ± 17.0 |
| Subject C | 19.8 ± 3.4 | 08.3 ± 6.3 | 20.0 ± 10.0 |
| Subject D | 18.9 ± 1.5 | 5.6 ± 7.3 | 27.6 ± 4.1 |
| Subject E | 15.3 ± 5.2 | −0.9 ± 9.6 | 46.6 ± 11.1 |
| Average | 17.0 ± 9.4 | −5.7 ± 12.0 1 Hz vs NoS | 36.1 ± 12.7 40 Hz vs |
| | | p < 0.05 a Hz vs 30 Hz | NoS p < 0.10 |
| | | p < 0.001 | |

TABLE 4
At the end of the assessment phase, the change in detrusor pressure in
five subjects during micturition attempts compared to the pre-attempt
baseline. Positive numbers indicate an increase in the pressure during
the attempts while negative numbers indicate a decrease in the
pressure during the attempts. Notice that high frequency stimulation
(30 Hz) did not change in detrusor pressure during attempted
micturition when compared to the no-stimulation/baseline; low frequency
stimulation (1 Hz), on the other hand, significantly increased
detrusor pressure compared to the non-stimulated and 30 Hz conditions.

| Δ P det | No | Low Frequency | High Frequency |
|---|---|---|---|
| (mmHg) | Stimulation | (1 Hz) | (30 Hz) |
| Subject A | 19.4 ± 2.2 | 38.8 ± 0.9 | 24.5 ± 4.5 |
| Subject B | 26.1 ± 1.5 | 28.2 ± 3.7 | 0.6 ± 1.3 |
| Subject C | 13. ± 0.6 | 30.4 ± 4.3 | 1.3 ± 0.9 |
| Subject D | 3.0 ± 1.5 | 49.4 ± 19.5 | 1.6 ± 0.8 |
| Subject E | −1.1 ± 0.7 | 58.5 ± 9.4 | −0.1 ± 0.7 |
| Average | 9.7 ± 12.2 | 41.1 ± 12.81 Hz vs Nos | 5.6 ± 10.6 30 Hv vs |
| | | p < 0.001 1 Hz vs | NoS p = 0.50 |
| | | 30 Hz p < 0.001 | |

Based on the BCR response, the evoked EMG activity and the responses of urethral and detrusor pressures, only 1 Hz TMSCS was used for weekly TMSCS during the treatment period.

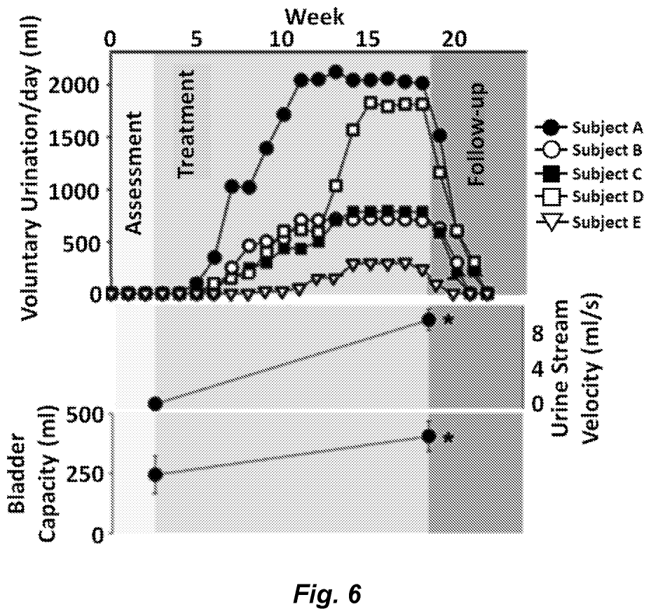

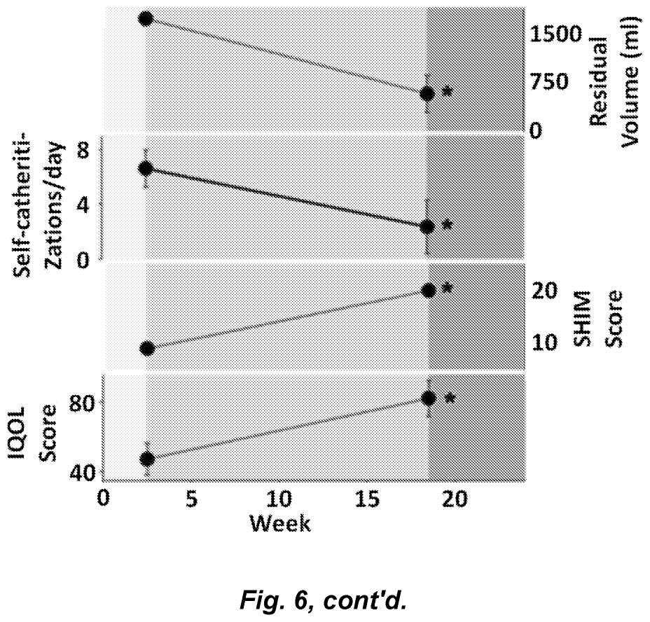

Bladder function before, during and after TMSCS All five subjects achieved at least some volitional urination following 16 weeks of bladder rehabilitation with TMSCS (FIG. 6). No subject achieved volitional urination until at least 4 weekly TMSCS treatments had been given, and the capacity to urinate voluntarily was restored in all 5 subjects on average 5.6±1.5 weeks after TMSCS was begun. The capacity to urinate voluntarily was maintained throughout the 16-week treatment period.

Daily self-catheterization decreased from 6.6 times per day at baseline to 2.4 times per day at the conclusion of the 16-week bladder rehabilitation (p=0.04). Based on urodynamic studies conducted at the end of the TMSCS treatment, the average volume of urine generated voluntarily increased from 0 cc/day to 1120 cc/day (p=0.03), and the subjects were able to generate significant urine stream velocities, which rose on average from 0 cc/sec to 9.3 cc/sec (p<0.001). The bladder capacity increased from 244 ml to 404 ml (p=0.02). Sexual function also improved from 9 to 20 as measured by Sexual Health Inventory for Men (SHIM) (p=0.0003). The subjects enjoyed a much higher quality of life; the average i-QOL score rose from 47 to 82 (p=0.007, FIG. 5). While all five subjects had improved bladder function and were able to achieve volitional micturition, their responses to TMSCS varied (the responsiveness order was A>D>B=C>E). This variation did not appear to be the result of differences in their AIS. (Table 2).

The average time that volitional micturition was maintained after the sham stimulation began was 3.2±0.8 weeks. Follow-up diary entries confirmed that the ability to void voluntarily rapidly decayed in all subjects after the cessation of effective TMSCS, and no subject maintained the capacity for voluntary micturition five weeks after the last effective stimulation.

### Discussion

Voluntary micturition requires complex, orchestrated neuromuscular control of the urinary bladder by sensory, motor and autonomic systems. During voluntary micturition, sympathetic inhibition of bladder contraction is withdrawn, parasympathetic activation of the detrusor contraction emerges to increase vesicular pressure, and contraction of the urethral sphincter is inhibited to allow urine to flow out of the bladder. This control is achieved through fronto-pontine-spinal cord projections to parasympathetic ganglia in the abdomen and to sympathetic and somatic neurons in the caudal spine. In individuals with SCI, coordination among parasympathetic, sympathetic and somatic nerve activities is lost: bladder pressure is elevated, but the bladder cannot be completely emptied because contraction of the external sphincter is not inhibited. Patients with SCI must perform multiple bladder self-catheterizations each day to evacuate urine and to prevent kidney injury due to high pressure; which increase the risk and frequency of infection and traumatic injury to the urethra. Any decrease in catheterization frequency, which was achieved in all study subjects, represents a potential decrease in complications associated with catheterization.

Isolated regions of lumbosacral spinal cord contain circuits that are capable of carrying out complex motor activities (Lu et al. (2015) Front. Molecular Neurosci. 8, 25, (2015); Sugaya & De Groat (1994) Am. J. Physiol., 266: R658-667). Furthermore, spinal cord injury in most motor complete, AIS A and B SCI subjects is not anatomically complete, and many spinal circuits remain intact, especially those below the level of the spinal cord injury (Heald et al. (2017) Neurorehabil. Neural Repair, 31: 583-591). In both animal and human subjects with chronic paralysis from SCI, motor movements have improved after invasive, epidural, electrical stimulations (Harkema et al. (2011) Lancet, 377: 1938-1947; Angeli et al. (2014) Brain: J. Neurol. 137: 1394-1409; Lu et al. (2016) Neurorehabil. Neural Repair, 30: 951-962). In this study, we hypothesized that the spinal micturition circuit remains intact in subjects with SCI, and since this circuit is semiautonomous, we should be able to enhance activation of patterned muscle activities controlled by these circuits and activate or modulate them using TMSCS over the thoracolumbar spine. The mechanism of action appears to be similar to the use of stimulation to improve upper extremity function in which the threshold of motor circuit activation is diminished to enable volitional, coordinated agonist-antagonist muscle activity (Alam et al. (2017) Exp. Neurol., 291: 141-150). Voluntary bladder control was restored to some extent by TMSCS in all five individuals with chronic SCI. Four out of five subjects (80%) were able to decrease the frequency of self-catheterization by at least 50%. One subject was able to void normally without any self-catheterization while another subject only needed one catheterization each day (Table 2).

Other attempts to restore urination in SCI patients by stimulating multiple peripheral nerves, specifically the pudendal, pelvic, hypogastric and tibial nerves (Schneider et al. (2015) Europ. Urol. 68: 859-867; Kennelly et al. (2011) J. Spinal Cord. Med. 34: 315-321; Spinelli et al. (2005) Neurology & Urodynam. 24: 305-309), did not consistently improve bladder function. Furthermore, sacral nerve modulation requires electrode implantation, which is invasive and risky (Zeiton et al. (2016) Int. J. Colorect. Dis. 31: 1005-1010; Eldabe et al. (2015) Complications of Spinal Cord Stimulation and Peripheral Nerve Stimulation Techniques: A Review of the Literature. Pain medicine (Malden, Mass.)). TMSCS differs in that it is non-invasive and painless in patients with SCI. In addition, TMSCS provides more consistent and effective bladder emptying than existing epidural stimulation of selected peripheral nerves (Bartley et al. (2013) Nat. Rev. Urology 10, 513-521; Brindley, (1974) J. Physiol., 237: 15 p-16 p; Van Kerrebroeck et al. (1996) J. Urol. 155: 1378-1381).

We believe that TMSCS allowed volitional activation of a coordinated pattern of parasympathetic withdrawal and sympathetic activation and somatic muscle inhibition as demonstrated in urodynamic studies. While the precise mechanism of TMSCS remains unknown, the coordinated activity of detrusor and sphincter muscles suggests that TMSCS works by activating or enhancing activation of central pattern generating circuits within the lumbosacral spinal cord and does not rely solely on activation of motor neurons or peripheral nerves. This hypothesis receives further support from the divergent responses to TMSCS at 1 Hz and 30 Hz: 1 Hz TMSCS resulted in decreased urethral pressure, increased detrusor pressure and micturition, as opposed to 30 Hz TMSCS, which increased urethral pressure, decreased detrusor pressure and enhanced urine storage within the bladder. The different stimulation frequencies elicited different bladder behaviors as if different central pattern generators (CPGs) or different aspects of a micturition CPG were activated. These divergent responses suggest that TMSCS may be applicable to a broader range of conditions such as hyperactive bladder, which may benefit from higher frequency stimulation.

We selected patients with detrusor-sphincter dyssynergia specifically because this is the group of SCI patients most recalcitrant to treatment. A regular schedule of self-catheterization prevents ureteral reflux and the development of obstructive uropathy and chronic renal failure, but frequent catheterization has risks of its own: infection, creation of false passages, urethral stricture (Prieto et al. (2015) Neurology & Urodynam. 34: 648-653; Bolinger et al. (2013) J. Wound, Ostomy, Continence, Nursing, 40: 83-89), a reduced quality of life, and a loss of independence. Improving quality of life is our ultimate goal using TMSCS, but this cannot be achieved if the risk of ureteral reflux and chronic renal failure increases. Therefore, any benefits of TMSCS, such as a more physiological voiding sequence with low storage pressures and increased bladder capacity and a better coordination of increased detrusor compliance and reduced external sphincter pressures that enable unobstructed voiding in a low pressure system, will be beneficial in the long term only if ureteral reflux is not increased. Video urodynamics performed at the initiation and termination of our study demonstrated no evidence of reflux. While this was a proof of concept, pilot study with patients followed for 16 weeks, additional studies are needed in an expanded cohort with extended follow-up to ensure that stable bladder and renal function are maintained when TMSCS is used to increase voluntary micturition and reduce the frequency of self-catheterization.

The BCR is a polysynaptic reflex, and BCR amplitudes in our subjects were 10 to 100 times larger at baseline than in normal individuals. Hyperactivity of the BCR may be analogous to the hyperactivity of tendon reflexes following SCI and suggests that subjects with SCI have decreased supraspinal inhibition of the BCR. During low frequency TMSCS, the amplitude of the BCR decreased, from which we infer that TMSCS induced greater inhibition of the BCR. Magnetic stimulation may achieve these effects by modulation of spinal interneurons via dorsal root ganglion or dorsal column stimulation, which is a putative mechanism of action for epidural spinal cord stimulation (Ramasubbu & Flagg (2013) Curr. Pain Headache Rep. 17: 315), or TMSCS may modulate responses within the sympathetic chain and sacral parasympathetic centers and facilitate the process of micturition.

Improvements in urinary function were not instantaneous; progressive improvement became apparent over the course of the study. Initially, simultaneous measurements of urethral and bladder pressures during volitional urination attempts revealed little (if any) sustained bladder contraction and persistently elevated urethral pressures, but after completion of at least 4 weeks of effective TMSCS, subjects became better able to generate sustained bladder contractions although detrusor-sphincter dyssynergia persisted (increased bladder pressures, but also increased urethral pressures, which prevented bladder emptying). At the end of the 16-week rehabilitation period, subjects were able to produce voluntary, coordinated bladder contractions with high detrusor pressures and reduced urethral pressures. Since bladder pressure exceed urethral pressure, urine flow velocity was increased and significantly higher urine volumes were achieved (FIG. 3).

Our subjects were able to urinate voluntarily in between treatment sessions when magnetic stimulation was not present. We believe that TMSCS persistently raised the activation state (or reduced inhibition) of the micturition circuit so that residual neural pathways between the supraspinal micturition centers and lumbosacral micturition central pattern generators were re-invigorated, which is consistent with previous findings using epidural stimulation to enhance recovery of motor function (Lu et al. (2016) Neurorehabil. Neural Repair, 30: 951-962). Restoration of voluntary micturition required repetitive TMSCS over at least 4 weeks. The benefits of epidural electric stimulations on motor function also required 3-5 sessions/weeks before improvements in motor functions were seen (Id.). Once supraspinal to spinal communication had been restored or re-enabled by TMSCS, it remained enabled so long as the subject received some minimal amount of TMSCS during each weekly treatment session, but the benefits of TMSCS were not permanent. All subjects lost the ability to control micturition soon after the termination of effective TMSCS (FIG. 6). The temporal dynamics of the onset and offset of benefit of TMSCS are consistent with remodeling of the spinal circuitry in which some relatively slow neuronal or circuit remodeling is required to re-establish effective synaptic or supraspinal communication (Boulis et al. (2013) Neurosurgery, 72: 653-661; Vallejo et al. (2016) Neuromodulation, 19: 576-586; Ryge et al. (2010) BMC Genomics, 11: 365), and some aspect of TMSCS was necessary between periods of volitional bladder emptying to maintain the integrity of communication between supraspinal and lumbar micturition circuits. The once weekly treatment interval and stimulation protocol represent a surprisingly small recurrent input to maintain volitional micturition, but this schedule is feasible for patients, and TMSCS could be administered in weekly physical therapy sessions at low cost. In any event, neuronal plasticity or remodeling are well recognized in TMS studies, specifically with low frequency (1 Hz) stimulation (O'Shea et al. (2007) Neuron, 54: 479-490; Lee et al. (2003) J. Neurosci. 23: 5308-5318). These results and our study of hand function (Lu et al. (2016) Neurorehabil. Neural Repair, 30: 951-962) provide two examples of the capacity of neuromodulation of spinal circuits to enable volitional control of motor functions below the level of SCI.

The responses to TMSCS varied among our five subjects. While we do not have a precise explanation for this, we know that the variation was not a result of differences among the AIS (Subject A, B, E were all category A, but subject A improved much more than the other two). The reasons for the variation are likely multifactorial, but perhaps most importantly, our subjects have variable amounts of residual spinal function. The current AIS is not sensitive to the subtleties of residual spinal functions among subjects.

The main limitations of our study are its small size and the lack of proof of the actual mechanism of action. As this is a pilot study, we plan to continue to expand the study and enroll additional subjects. Further studies will focus on the molecular and cellular processes that follow magnetic stimulation to investigate the precise mechanism of action of magnetic stimulation.

### Methods

### Subject Selection

We conducted a pilot, prospective, interventional study in five subjects. All aspects of the study were approved by the UCLA IRB (IRB# 14-000932) and filed with ClinicalTrials.gov (registration number: NCT02331979, date of registration: Jun. 1, 2015). All methods were performed in accordance with the relevant guidelines and regulations as stipulated by UCLA IRB. Informed consent was obtained prior to subject participation. The inclusion criteria for the study were male age 18-75, a stable American Spinal Injury Association Impairment Scale (AIS) A/B, motor complete spinal cord injury between spinal levels C2-T8 present for greater than 1 year, and a documented history of neurogenic bladder requiring intermittent catheterization. Each subject was required to have at least three prior urodynamic studies to confirm the diagnosis of neurogenic bladder with detrusor sphincter dyssynergia (DSD), which was diagnosed with urodynamic study in which a rise in detrusor pressure and concomitant needle EMG activity and rise in urethral pressure were demonstrated (see Tables 3 and 4). Patients with a history of autonomic dysreflexia were excluded from the study. Any patient who was ventilator dependent, abusing drugs, had musculoskeletal dysfunction (i.e., unstable fractures), cardiopulmonary diseases, active infections or ongoing depression requiring treatment, or had previous exposure to and use of spinal cord stimulation was excluded from the study. Patients with a history of bladder botox injection or bladder/sphincter surgeries were excluded. Five subjects were recruited and completed the study. There was no subject attrition.

### Intervention

Each study subject underwent baseline urodynamic testing (UDS) at the beginning of the study to confirm the diagnosis of a neurogenic bladder with DSD and establish baseline bladder functions. The study was divided into three phases: an Assessment phase (2 weeks), a Treatment phase (16 weeks) and a Follow-up phase (6 weeks). During the Assessment phase, each subject underwent once/week transcutaneous magnetic spinal cord stimulation (TMSCS) at both 1 Hz (low) and 30 Hz (high) frequency (40-60% intensity) over the lumbar spine (described below). 1 Hz and 30 Hz were both administered during the assessment phase because the optimal stimulation frequency in human subjects was unknown to us prior to this study. The two frequencies were administered in random order. These frequencies was chosen based on previous results in animals in which low frequency stimulation promoted and high frequency stimulation inhibited micturition in animals with SCI (Alam et al. (2017) Exp. Neurol., 291: 141-150). At the conclusion of the Assessment phase, each subject underwent another UDS to determine the better stimulation frequency (the characteristics of optimal stimulation are defined below). Once the better frequency was established (and it turned out that 1 Hz was better than 30 Hz stimulation in all five subjects), each subject entered the treatment phase of the study and received weekly transcutaneous lumbar spinal cord magnetic stimulation for a total of 16 weeks (described below). This 16-week period of TMSCS constituted bladder rehabilitation. Each subject received non-video urodynamic testing once every four weeks during the treatment phase to monitor progress and insure that bladder function was not further impaired. After the initial four-week stimulation period, each subject was asked to attempt volitional urination for 5-10 minutes prior to bladder catheterization. The subjects were instructed to keep the environment quiet, relax and focus on voiding. Specifically, they were instructed to perform no straining/Valsalva maneuver, external compression by Crede maneuver, reflex triggering by tapping, anal stretch, or pushing. Attempts were limited to 10 minutes. Each subject was given a urine/stool specimen collection pan (Medline DYND36600H, Mundelein, Ill.) to collect any volitional urinary output. In order to prevent potential urinary retention, the subjects were asked to self-catheterize after the volitional attempt and to record the catheterization output. The urinary output and the volume of the residual urine in the bladder (collected after attempted void by each patient's routine bladder catheterization) were recorded in a diary after every attempt to urinate voluntarily. Each subject was also asked to record any other changes that he may have noticed in the diary throughout the study period. During the follow-up period, sham transcutaneous magnetic stimulation (sham) was employed at reduced intensity (5%), which replicated the auditory, partial sensory and mechanical cues of real stimulation. Each subject was instructed to continue to attempt to urinate voluntarily as he had during the treatment phase, and each subject continued to keep a detailed urological lifestyle diary until the end of the follow-up phase (FIG. 2).

Each subject was also given an incontinence quality of life (iQOL) questionnaire to complete prior to the start of the study and at the end of the 16-week treatment stimulation. iQOL has been validated in multiple urological quality of life studies in patients with SCI (Patrick et al. (1999) Eur. Urol. 36: 427-435; Jo et al. (3026) Pain Physician, 19: 373-380). Sexual functions were assessed by Sexual Health Inventory for Men (SHIM) questionnaire (Barbonetti et al. (2012) J. Sex. Med. 9: 830-836) at the beginning of the study and at the end of 16-week treatment phase.

### Blinding

The state of knowledge at the start of the study, in which we did not know the effective parameters of stimulation to effect micturition, precluded a randomized trial. Therefore, we conducted a single arm study in which each subject acted as his own control. Additionally, a sham phase was conducted at the end after stimulation because there was exposure to stimulation during initial Assessment Phase (we had no initial, baseline, non-stimulated collection period), and we were unsure of the wash-out period for this exposure due to the pilot nature of this study. However, subjects and experimenters were blinded throughout the process in the assessment, treatment and follow-up phases. Given that these spinal cord injury patients have diminished/no sensation due to their injuries, The subjects did not feel any sensations at 1 Hz or 30 Hz at the level of stimulation used during treatment as they have muted sensory capacity due to their spinal cord injury. We purposefully selected a relatively low intensity stimulus to avoid any painful sensations, and the stimulation level was below the sensory and motor threshold. The subjects did hear a “click” during each stimulation (especially at 1 Hz when the click was very predicable). This auditory cue was re-created during sham stimulation as well. Approximately from T11-L3 level. The coil dimensions are 172×92×51 mm in a figure-of-eight formation with two rings each 75 mm in diameter. The target (focality) is the center of the figure of eight, which in our case is T12-L1 area, which overlies the conus medullaris in humans. We used a research coil (with identical sham and treatment faces), which allowed blinding of both experimenter and subject; thereby double blinding the follow-up phase of the study. The staff member responsible for controlling of the stimulator and the dose of stimulation was not blinded as the stimulation parameters were manipulated during the various phases of the study; however, this person did not interact with the subject (he sat behind a curtain), and each staff member was instructed to follow the same script when administering the various tasks regardless of the particular stimulation values used. To assess integrity of blinding, we asked subjects at the conclusion of the study what each study phases consisted of; their responses were no better than chance.

### Urodynamic Testing

We employed a commercially available urodynamic machine (Laborie Aquarius® XT, Laborie International, Mississauga, ON, Canada). Prior to the urodynamic testing, each subject emptied his bladder by direct catheterization. The volume of urine was recorded. The patient was then placed in a supine position and a triple lumen catheter (TLC-7M, Laborie International, Mississauga, ON, Canada) was inserted. Two needle recording electrodes (1512A-M, Laborie International, Mississauga, ON, Canada) were inserted bilaterally into the perineal muscles approximately halfway between the base of the scrotum and anus and 1 cm lateral to the midline. An EMG grounding pad was placed on the knee joint. A rectal catheter (RPC-9, Laborie International, Mississauga, ON, Canada) was inserted to record abdominal pressure. The subject was next placed in a left decubitus position. A condom catheter was used to collect any urine output, which was directed through a funnel into a graduated cylinder (DIS173, Laborie International, Mississauga, ON, Canada) on a scale (UROCAP IV, Laborie International, Mississauga, ON, Canada) to record the volume of urine produced and the stream velocity.

### Transcutaneous Magnetic Stimulation

A MagVenture Magnetic Stimulator (MagPro R30, Atlanta, Ga.) with an active/placebo figure-8 research coil (Cool-B65 A/P Coil) was used for all transcutaneous magnetic stimulation sessions. The spinous processes of the lower vertebrae in each subject were palpated, and thoracic 11 to lumbar 4 vertebrae were marked. The coil was centered along the midline at the L1 vertebral level during the stimulation and oriented such that the magnetic field generated was parallel to the spinal cord (rostral-caudal). We used trains of biphasic, single pulse (duration 250 μs), continuous, magnetic stimulation. Each stimulation session consisted of three 4-min continuous stimulation periods with a 30 second break between each stimulation period for a total of 13 minutes (a total of 12 minutes of stimulation plus 1 minute of breaks). For the first two weeks, each subject underwent stimulation at 1 Hz and 30 Hz frequencies (week one: 1 Hz/30 Hz/1 Hz, and week two: 30 Hz/1 Hz/30 Hz) until the better frequency was determined for the patient at the first follow-up UDS after the 2 week of stimulation. The frequency of 1 Hz and 30 Hz was selected based on our previous work in animals and humans (Lu et al. (2016) Neurorehabil. Neural Repair, 30: 951-962; Gad et al. (2014) PloS one 9: e108184). Changes in urethral (directly measured) and detrusor (vesicular-abdominal) pressures during micturition attempts were measured during both low frequency stimulation (1 Hz) and high frequency stimulation (30 Hz). The stimulation frequency that resulted in the combination of increased detrusor pressure and decreased urethral pressure during attempted micturition (hence, promoting bladder emptying) was selected as optimal. The intensity of stimulation was set 20% below the intensity that elicited local paraspinal muscular contraction for each subject (since muscle contractions would have unmasked the double blinding). This stimulation intensity was usually around 40-50% of the maximal field strength of 2 Tesla. Once the optimal frequency was determined, all subjects received the optimal stimulation frequency only at a constant intensity for the remaining 16, weekly bladder rehabilitation sessions.

### Electrophysiology

At the end of the assessment phase, the following electrophysiological data were obtained on each subject before, during and after low frequency (1 Hz) and high frequency (30 Hz) transcutaneous magnetic stimulation: bulbocavernosus reflex (BCR), electromyography (EMG) and spinal evoked potentials (SEP) bilaterally in the pelvic floor and in the vastus lateralis, gastrocnemius, gluteus and quadriceps femoris muscles.

Pelvic floor EMGs were obtained using needle electrodes (Laborie 1512A-M, Laborie International, Mississauga, ON, Canada). All other muscle EMGs were obtained with 1 inch surface pad electrodes (MultiBioSensors, El Paso, Tex.).

The BCR was obtained by using ring stimulating electrodes (Cadwell 302243-200, Cadwell Industries, Kennewick, Wash.) that were stimulated with a monophasic electric pulse at 1.5 Hz, pulse width 0.2 ms, and intensity at three times the sensory threshold (or 35 mA if the subject had no sensation). At least 100 pulses were given in each BCR test session. Recording, amplification and digitization of all data were done using an RZ2 amplifier and a PZ5-32 TDT digitizer (Tucker Davis Technologies, Alachua, Fla.) with a 60 Hz notch filter and band pass filtering to exclude frequencies <3 Hz and >200 Hz.

### Data Analysis

The primary outcome was voluntary urination volume per day. Pre-specified secondary outcomes included urine stream flow rate, bladder capacity, catheterizations per day, sexual health inventory for men (SHIM), and urinary incontinence quality of life scale (iQOL). All electrophysiological data from the TDT system (Tucker Davis Technologies, Alachua, Fla.) were exported to a computer and analyzed using MatLab (Matlab2015b, MathWorks, Natick, Mass.).

The BCR amplitudes and latency were calculated for every single electrical pudendal stimulation. Spinal evoked potentials (if present) were identified in the continuous recording of lower extremity EMGs.

Urodynamic data were exported from the Laborie system and analyzed using Microsoft Excel (Excel2010, Microsoft, Redmond, Wash.). The changes in urethral pressure (P urethra) and detrusor pressure (P detrusor) were measured and compared during baseline and during attempted micturition. Statistical significance was assessed with Analysis of Variance (ANOVA) and paired Student's T-tests and the Bonferroni correction for multiple preplanned comparisons, when appropriate, using R 3.25 (www.r-project.org) and Graphpad Prism (Graphpad Software, La Jolla, Calif.), respectively.

### Example 2

### Transcutaneous Magnetic Spinal Cord Stimulation for Neurogenic Bladder

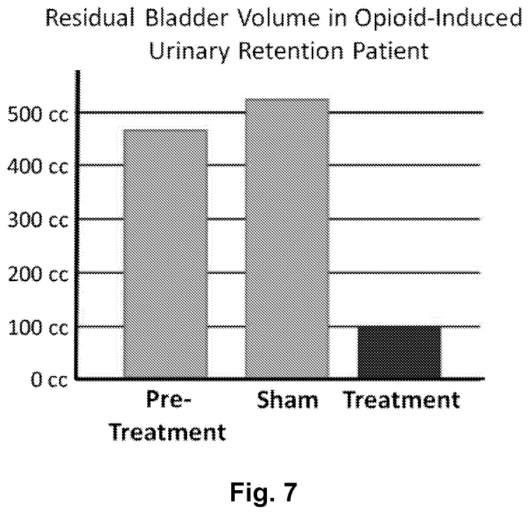

FIG. 7 shows the residual bladder volume in a post-operative opioid-induced urinary retention patient treated with non-invasive magnetic spinal cord stimulation. A 68 year-old male patient with urinary retention after surgery reported inability to urinate. A pre-treatment bladder ultrasound demonstrated 420+cc of residual bladder volume. Over the course of 2 hours of attempted unsuccessful voluntary urination, bladder volume increased to 500+ cc. The subject was treated with transcutaneous magnetic stimulation at L1/2 spinal vertebral body region (conus medullaris) for 15 minutes (60% intensity of 2 Tesla field strength; biphasic, single pulse, 250 μs). After conclusion of the treatment session, patient was able to void voluntarily with approximately 100 cc of residual bladder volume. Subsequently, the patient demonstrated another volitional attempt to void voluntarily prior to discharge.

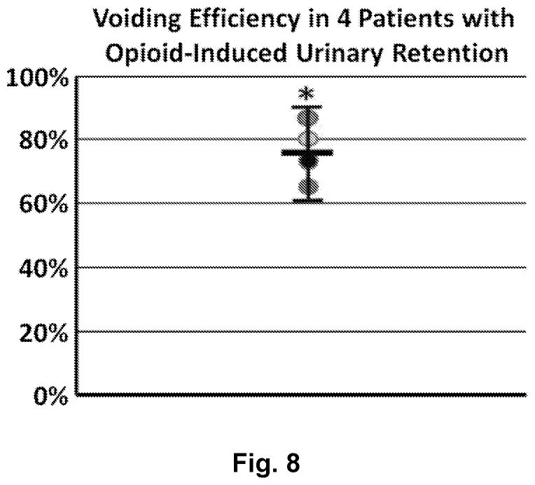

FIG. 8 shows the bladder voiding efficiency in four patients with opioid-induced urinary retention that were treated with non-invasive magnetic spinal cord stimulation. Four male patients with urinary retention after surgery presented with inability to urinate. Bladder ultrasound was used to document bladder volume and characterize urinary efficiency, where urinary efficiency was defined as voided volume/(voided volume+residual volume). Treatment was performed with transcutaneous magnetic stimulation at L1/2 spinal vertebral body region (conus medullaris) for 15 minutes and repeated up to three times (1 Hz, 50-70% intensity of 2 Tesla field strength; biphasic, single pulse, 250 us). Bladder voiding efficiency improved to over 60% for all patients following treatment, including nearly 90% bladder voiding efficiency for one patient. *P=0.006, by paired t-test.

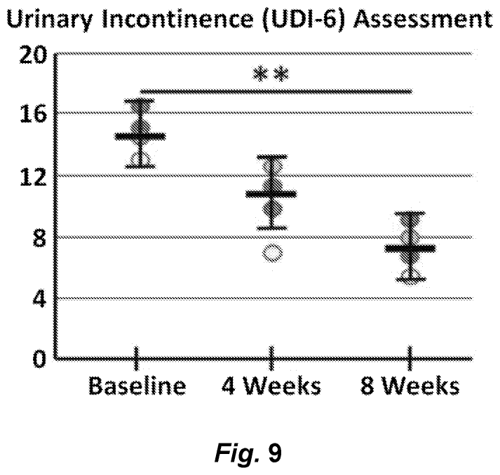

FIG. 9 shows the results of an assessment of incontinence in patients without brain or spinal cord injury, treated with non-invasive magnetic spinal cord stimulation. Three 3 female patients with stress incontinence were treated with non-invasive spinal cord stimulation to L1/2 (weekly 15 minute treatment, 30 Hz, 60-75% intensity 60-70% intensity of 2 Tesla field strength; biphasic, single pulse, 250 μs) and assessment of urinary function was made at 4 and 8 weeks. Assessment was made by Urinary Distress Inventory, Short Form (UDI-6). **P=0.004 by one-way ANOVA; **, P<0.01 by post-hoc Tukey). As illustrated in FIG. 9, non-invasive magnetic stimulation provided a significant improvement (lower UDI-6 score) in treated patients.

### Example 3

### Transcutaneous Magnetic Spinal Cord Stimulation for Post-Operative Ileus

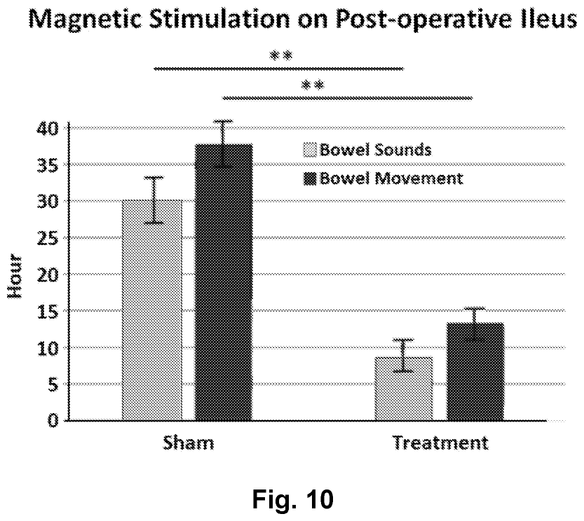

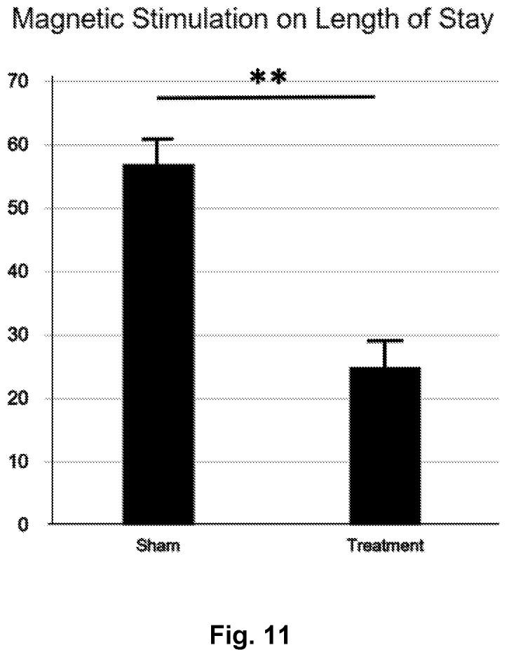

FIG. 10 shows the results for seven patients that underwent anterior lumbar interbody fusion (ALIF) surgery for the treatment of spinal degeneration, a procedure which required abdominal retroperitoneal surgical approach that induced post-operative ileus (e.g., constipation). Out of these patients, three patients were treated with magnetic stimulation (Treatment) and four patients were treated with sham stimulation (Sham), then assessed for post-operative bowel sounds and bowel movement. Specifically, after surgery, assessment of bowel sounds was performed with abdominal auscultation performed by nurses at hourly intervals. Stimulation was applied to the three treated patients at the conus medullaris of the spinal cord every 2 hours, with each treatment session having a duration of 15 minutes of stimulation at 1 Hz, 60-75% intensity of 2 Tesla field strength; biphasic, single pulse, 250 μs. The conus medullaris was identified in each patient by pre-operative MRI, and localization was identified by AP X-ray correlated to surface landmark of spinous process interspace and localized to L1-L3 vertebral body levels among this cohort. As shown in FIG. 10, magnetic stimulation significantly reduced the time to bowel sounds and bowel movement for the treated patients relative to the patients treated with sham stimulation, thereby resolving the post-operative ileus. **P<0.05 by paired t-test.

Post-operative ileus generally is associated with increased duration of hospitalization, due to lack of bowel movement. Subjects from FIG. 10 were analyzed for length of stay. As shown in FIG. 10, patients treated with magnetic stimulation significantly decreased length of stay as compared to sham treated patients. **P<0.05 by paired t-test.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of facilitating voiding or control of bladder and/or bowel in a subject with dysfunctional bladder and/or bowel function where said subject does not have a spinal cord or brain injury, said method comprising:
providing magnetic stimulation of the spinal cord at a location, frequency and intensity sufficient to facilitate voiding or control of bladder and/or bowel.

2. The method of claim 1, wherein said dysfunctional bladder and/or bowel comprises neurogenic bladder dysfunction.

3. The method of claim 1, wherein said dysfunctional bladder and/or bowel comprises post-surgical constipation.

4. The method of claim 1, wherein said dysfunctional bladder and/or bowel comprises narcotic-induced constipation.

5. The method of claim 4, wherein said dysfunctional bladder and/or bowel comprises opioid constipation.

6. The method of claim 1, wherein said dysfunctional bladder and/or bowel comprises dysfunction induced by an inflammatory stimulus, such as trauma or infection.

7. The method of claim 1, wherein said dysfunctional bladder and/or bowel comprises pregnancy associated bladder and/or bowel dysfunction.

8. The method of claim 1, wherein said dysfunctional bladder and/or bowel is associated with a condition selected from the group consisting of meningomyelocele, diabetes, AIDS, alcohol abuse, vitamin B12 deficiency neuropathies, herniated disc, damage due to pelvic surgery, syphilis, and a tumor.

9. The method according to any one of claims 1-8, wherein said method comprises facilitating voiding or control of bladder and/or bowel by providing magnetic stimulation of the spinal cord at a location, frequency and intensity sufficient to facilitate voiding or control of the bladder and/or bowel.

10. The method according to any one of claims 1-9, wherein said magnetic stimulation comprises stimulation at a frequency ranging from about 0.5 Hz up to about 15 Hz to induce micturition.

11. The method of claim 10, wherein said magnetic stimulation is at a frequency of about 1 Hz.

12. The method according to any one of claims 1-9, wherein said magnetic stimulation comprises stimulation at a frequency from about 20 Hz up to about 100 Hz to stop or prevent micturition.

13. The method of claim 12, wherein said magnetic stimulation is at a frequency of about 30 Hz.

14. The method according to any one of claims 1-13, wherein said magnetic stimulation comprises magnetic pulses ranging in duration from about 5 μs, or from about 10 μs, or from about 15 μs, or from about 20 μs up to about 500 μs, or up to about 400 μs, or up to about 300 μs, or up to about 200 μs, or up to about 100 μs. or up to about 50 μs.

15. The method of claim 14, wherein said magnetic pulses are about 25 μs in duration.

16. The method according to any one of claims 1-15, wherein said magnetic stimulation is monophasic.

17. The method according to any one of claims 1-16, wherein a single treatment of said magnetic stimulation comprises 1, or 2, or 3, or 4, or 5, or 6, or 7, or 8, or 9, or 10 or more continuous stimulation periods.

18. The method of claim 17, wherein a single treatment of said magnetic stimulation comprises about 3 continuous stimulation periods.

19. The method according to any one of claims 17-18, wherein said continuous stimulation periods range in duration from about 10 sec, or from about 20 sec, or from about 3 sec or from about 40 sec, or from about 50 sec, or from about 1 min, or from about 2 minutes up to about 10 minutes, or up to about 8 minutes, or up to about 6 minutes.

20. The method of claim 19, wherein said continues stimulation periods are about 4 minutes in duration.

21. The method according to any one of claims 17-20, wherein a delay between continuous stimulation periods ranges from about 5 sec, or from about 10 sec, or from about 15 sec, or from about 20 sec up to about 5 minutes, or up to about 4 minutes, or up to about 3 minutes, or up to about 2 minutes, or up to about 1 min, or up to about 45 sec, or up to about 30 sec.

22. The method of claim 21, wherein a delay between continuous stimulation periods is about 30 sec.

23. The method according to any one of claims 17-22, wherein said treatment is repeated.

24. The method of claim 23, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days.

25. The method according to any one of claims 23-24, wherein the treatment is repeated over a period of at least 1 week, or at least two weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 4 months, or at least 5 months, or at least 6 months, or at least 7 months, or at least 8 months, or at least 9 months, or at least 10 months, or at least 11 months, or at least 12 months.

26. The method according to any one of claims 1-25, wherein treatment of said subject with said magnetic stimulation facilitates volitional voiding at a later time without magnetic stimulation.

27. The method according to any one of claims 23-26, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains volitional control of micturation.

28. The method of claim 27, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains their maximal volitional control of micturation.

29. The method of claim 27, wherein the frequency of treatment is reduced after the subject obtains volitional control of micturition.

30. The method of claim 28, wherein the frequency of treatment is reduced after the subject obtains maximal volitional control of micturition.

31. The method according to any one of claims 29-30, wherein the frequency of treatment is reduced to a level sufficient to maintain volitional control of micturition.

32. The method of claim 31, wherein the frequency of treatment is reduced to every three days, or to a weekly treatment, or to about every 10 days, or to about every 2 weeks.

33. The method according to any one of claims 1-32, wherein said magnetic stimulation is applied over the thoracic and/or lumbosacral spinal cord.

34. The method of claim 33, wherein said magnetic stimulation is applied over one or more regions selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, T12-T12, L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6.

35. The method of claim 33, wherein said magnetic stimulation is applied over a region between T11 and L4.

36. The method of claim 35, wherein said magnetic stimulation is applied over one or more regions selected from the group consisting of T11-T12, L1-L2, and L2-L3.

37. The method of claim 35, wherein said magnetic stimulation is applied over L1-L2 and/or over T11-T12.

38. The method of claim 35, wherein said magnetic stimulation is applied over L1.

39. The method according to any one of claims 1-38, wherein said magnetic stimulation is applied at the midline of spinal cord.

40. The method according to any one of claims 1-39, wherein said magnetic stimulation produces a magnetic field of at least about 1 tesla, or at least about 2 tesla, or at least about 3 tesla, or at least about 4 tesla, or at least about 5 tesla.

41. The method according to any one of claim 1-9, or 17-40, wherein said magnetic stimulation is at a frequency of at least about 0.5 Hz, 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz.

42. A method of facilitating voiding or control of bladder and/or bowel in a subject with a dysfunctional bladder and/or bowel function where said subject does not have a spinal cord or brain injury, said method comprising:
providing transcutaneous electrical stimulation of the spinal cord at a location, frequency and intensity sufficient to facilitate voiding or control of bladder and/or bowel.

43. The method of claim 42, wherein said dysfunctional bladder and/or bowel comprises neurogenic bladder dysfunction.

44. The method of claim 42, wherein said dysfunctional bladder and/or bowel comprises post-surgical constipation.

45. The method of claim 42, wherein said dysfunctional bladder and/or bowel comprises narcotic-induced constipation.

46. The method of claim 45, wherein said dysfunctional bladder and/or bowel comprises opioid constipation.

47. The method of claim 42, wherein said dysfunctional bladder and/or bowel comprises dysfunction induced by an inflammatory stimulus, such as trauma or infection.

48. The method of claim 42, wherein said dysfunctional bladder and/or bowel comprises pregnancy associated bladder and/or bowel dysfunction.

49. The method of claim 42, wherein said dysfunctional bladder and/or bowel is associated with a condition selected from the group consisting of Meningomyelocele, Diabetes, AIDS, Alcohol abuse, Vitamin B12 deficiency neuropathies, Herniated disc, damage due to pelvic surgery, Syphilis, and a tumor.

50. The method according to any one of claims 42-49, wherein said method comprises facilitating voiding or control of bladder and/or bowel by providing transcutaneous electrical stimulation of the spinal cord at a location, frequency and intensity sufficient to facilitate voiding or control of the bladder and/or bowel.

51. The method according to any one of claims 42-50, wherein said transcutaneous electrical stimulation comprises stimulation at a frequency of at least about 1 Hz, or at least about 2 Hz, or at least about 3 Hz, or at least about 4 Hz, or at least about 5 Hz, or at least about 10 Hz, or at least about 20 Hz or at least about 30 Hz or at least about 40 Hz or at least about 50 Hz or at least about 60 Hz or at least about 70 Hz or at least about 80 Hz or at least about 90 Hz or at least about 100 Hz, or at least about 200 Hz, or at least about 300 Hz, or at least about 400 Hz, or at least about 500 Hz, and/or at a frequency ranging from about 1 Hz, or from about 2 Hz, or from about 3 Hz, or from about 4 Hz, or from about 5 Hz, or from about 10 Hz, or from about 10 Hz, or from about 10 Hz, up to about 500 Hz, or up to about 400 Hz, or up to about 300 Hz, or up to about 200 Hz up to about 100 Hz, or up to about 90 Hz, or up to about 80 Hz, or up to about 60 Hz, or up to about 40 Hz, or from about 3 Hz or from about 5 Hz up to about 80 Hz, or from about 5 Hz to about 60 Hz, or up to about 30 Hz. In certain embodiments the transcutaneous stimulation is at a frequency ranging from about 20 Hz or about 30 Hz to about 90 Hz or to about 100 Hz.

52. The method according to any one of claims 42-51, wherein the transcutaneous electrical stimulation is provided on a high frequency carrier signal.

53. The method of claim 52, wherein the high frequency carrier signal ranges from about 3 kHz, or about 5 kHz, or about 8 kHz up to about 30 kHz, or up to about 20 kHz, or up to about 15 kHz.

54. The method according to any one of claims 52-53, wherein the carrier frequency amplitude ranges from about 30 mA, or about 40 mA, or about 50 mA, or about 60 mA, or about 70 mA, or about 80 mA up to about 300 mA, or up to about 200 mA, or up to about 150 mA.

55. The method according to any one of claims 52-54, wherein said transcutaneous electrical stimulus is a high frequency stimulus at a duration ranging from about 0.1 up to about 2 ms, or from about 0.1 up to about 1 ms, or from about 0.5 ms up to about 1 ms, or for about 0.5 ms.

56. The method according to any one of claims 52-55, wherein the transcutaneous electrical stimulation comprises a 10 kHz stimulus repeated at 1-40 times per second.

57. The method according to any one of claims 42-56, wherein said transcutaneous electrical stimulus is applied for 1 to 30 s, or for about 5 to 30 s, or for about 10 to about 30 s.

58. The method according to any one of claims 42-57, wherein said transcutaneous electrical stimulus is about 30 to about 100 mA.

59. The method according to any one of claims 52-58, wherein said transcutaneous electrical stimulus comprises a 10 kHz signal applied at 1 Hz.

60. The method according to any one of claims 42-59, wherein said transcutaneous electrical stimulus comprises a constant-current bipolar rectangular stimulus.

61. The method according to any one of claims 42-60, wherein said transcutaneous electrical stimulation comprises pulses ranging in duration from about 5 μs, or from about 10 μs, or from about 15 μs, or from about 20 μs up to about 2 ms, or up to about 1 ms, or up to about 2 ms, or up to about 500 μs, or up to about 400 μs, or up to about 300 μs, or up to about 200 μs, or up to about 100 μs. or up to about 50 μs.

62. The method of claim 61, wherein said pulses are about 1 ms in duration.

63. The method according to any one of claims 42-62, wherein a single treatment of said transcutaneous electrical stimulation comprises 1, or 2, or 3, or 4, or 5, or 6, or 7, or 8, or 9, or 10 or more continuous stimulation periods.

64. The method of claim 63, wherein said treatment is repeated.

65. The method of claim 64, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days.

66. The method according to any one of claims 64-65, wherein the treatment is repeated over a period of at least 1 week, or at least two weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 4 months, or at least 5 months, or at least 6 months, or at least 7 months, or at least 8 months, or at least 9 months, or at least 10 months, or at least 11 months, or at least 12 months.

67. The method according to any one of claims 42-66, wherein treatment of said subject with said transcutaneous electrical stimulation facilitates volitional voiding at a later time without transcutaneous electrical stimulation.

68. The method according to any one of claims 64-67, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains volitional control of micturation.

69. The method according to any one of claims 64-67, wherein said treatment is repeated daily, or every 2 days, or every 3 days, or every 4 days, or every 5 days, or every 6 days, or every 7 days, or every 8 days, or every 9 days, or every 10 days, or every 11 days, or every 12 days, or every 13 days, or every 14 days until the subject obtains their maximal volitional control of micturation.

70. The method according to any one of claims 64-67, wherein the frequency of treatment is reduced after the subject obtains volitional control of micturition.

71. The method according to any one of claims 64-67, wherein the frequency of treatment is reduced after the subject obtains maximal volitional control of micturition.

72. The method according to any one of claims 70-71, wherein the frequency of treatment is reduced to a level sufficient to maintain volitional control of micturition.

73. The method according to any one of claims 42-72, wherein said transcutaneous electrical stimulation is applied over one or more regions selected from the group consisting of T1-T1, T1-T2, T1-T3, T1-T4, T1-T5, T1-T6, T1-T7, T1-T8, T1-T9, T1-T10, T1-T11, T1-T12, T2-T2, T2-T3, T2-T4, T2-T5, T2-T6, T2-T7, T2-T8, T2-T9, T2-T10, T2-T11, T2-T12, T3-T3, T3-T4, T3-T5, T3-T6, T3-T7, T3-T8, T3-T9, T3-T10, T3-T11, T3-T12, T4-T4, T4-T5, T4-T6, T4-T7, T4-T8, T4-T9, T4-T10, T4-T11, T4-T12, T5-T5, T5-T6, T5-T7, T5-T8, T5-T9, T5-T10, T5-T11, T5-T12, T6-T6, T6-T7, T6-T8, T6-T9, T6-T10, T6-T11, T6-T12, T7-T7, T7-T8, T7-T9, T7-T10, T7-T11, T7-T12, T8-T8, T8-T9, T8-T10, T8-T11, T8-T12, T9-T9, T9-T10, T9-T11, T9-T12, T10-T10, T10-T11, T10-T12, T11-T11, T11-T12, T12-T12, L1-L1, L1-L2, L1-L3, L1-L4, L1-L5, L1-S1, L1-S2, L1-S3, L1-S4, L1-S5, L2-L2, L2-L3, L2-L4, L2-L5, L2-S1, L2-S2, L2-S3, L2-S4, L2-S5, L3-L3, L3-L4, L3-L5, L3-S1, L3-S2, L3-S3, L3-S4, L3-S5, L4-L4, L4-L5, L4-S1, L4-S2, L4-S3, L4-S4, L4-S5, L5-L5, L5-S1, L5-S2, L5-S3, L5-S4, L5-S5, S1-S1, S1-S2, S1-S3, S1-S4, S1-S5, S2-S2, S2-S3, S2-S4, S2-S5, S3-S3, S3-S4, S3-S5, S4-S4, S4-S5, and S5-S6.

74. The method of claim 73, wherein said transcutaneous electrical stimulation is applied over a region between T11 and L4.

75. The method of claim 74, wherein said transcutaneous electrical stimulation is applied over one or more regions selected from the group consisting of T11-T12, L1-L2, and L2-L3.

76. The method of claim 74, wherein said transcutaneous electrical stimulation is applied over L1-L2 and/or over T11-T12.

77. The method of claim 74, wherein said transcutaneous electrical stimulation is applied over L1.

78. The method according to any one of claims 42-77, wherein said transcutaneous electrical stimulation is applied at the midline of spinal cord.

79. The method according to any one of claims 1-78, wherein said subject is a subject without a neurodegenerative pathology.

80. The method of claim 79, wherein said subject does not have Parkinson's disease, Huntington's disease, Alzheimer's disease, amyotrophic lateral sclerosis (ALS), primary lateral sclerosis (PLS), and/or cerebral palsy.

81. A method of facilitating voiding or control of bladder and/or bowel in a subject with dysfunctional bladder and/or bowel function where said subject does not have a spinal cord or brain injury, said method comprising:
providing magnetic stimulation in combination with transcutaneous electrical stimulation at one or more locations, frequencies, and intensities sufficient to facilitate voiding or control of bladder and/or bowel.

82. The method of claim 81, wherein said method comprises providing magnetic stimulation to said subject using a method according to any one of claims 1-41 in combination with electrical stimulation using a method according to any one of claims 42-78.
